(19) United States
(12) Patent Application Publication
(10) Pub. No.: US 2024/0153585 A1
(43) Pub. Date: May 9, 2024

(54) METHOD

(71) Applicant: Société des Produits Nestlé S.A., Vevey (CH)

(72) Inventors: Rondo Middleton, CREVE COEUR, MO (US); James HOLZWARTH, Yverdon les Bains (CH); Lorane TEXARI, Morges (CH); Alix ZOLLINGER, Denges (CH)

(73) Assignee: Société des Produits Nestlé S.A., Vevey (CH)

(21) Appl. No.: 18/502,481

(22) Filed: Nov. 6, 2023

Related U.S. Application Data
(60) Provisional application No. 63/423,671, filed on Nov. 8, 2022.

(51) Int. Cl. G16B 30/10; C12Q 1/6806; C12Q 1/6874; G16H 20/60; G16H 50/30

(52) U.S. Cl. CPC G16B 30/10; C12Q 1/6806; C12Q 1/6874; G16H 20/60; G16H 50/30; C12Q 2600/154

(57) ABSTRACT

The present invention relates to a method of determining the contribution of a dog breed to a test dog genome, comprising: a) providing a DNA methylation profile from a sample obtained from the test dog; and b) determining the contribution of a dog breed to the test dog genome by comparing at least part of the DNA methylation profile of the test dog to reference DNA methylation profiles from different dog breeds.

### CROSS REFERENCE TO RELATED APPLICATION

This application claims priority to U.S. Provisional Application Ser. No. 63/423,671 filed Nov. 8, 2022 the disclosure of which is incorporated in its entirety herein by this reference.

### SEQUENCE LISTING

The instant application contains a Sequence Listing which has been submitted electronically in XML file format and is hereby incorporated by reference in its entirety. Said XML copy, created on Dec. 20, 2023, is named 18954-US—NP_SL.xml and is 218,196 bytes in size.

### FIELD OF THE INVENTION

The present invention relates to a method for determining the contribution of a dog breed to a test dog genome using a DNA methylation profile. The invention further relates to methods of selecting a dietary, lifestyle or pharmacological regime for the dog, for example to prevent or reduce the risk of a dog developing a disease, based on the contribution of at least one dog breed to the test dog genome determined from the DNA methylation profile.

### BACKGROUND TO THE INVENTION

The ability to determine information regarding the breed of a dog is desirable to inform about the dog's ancestry and to provide information regarding its general health and well-being, for example its disease susceptibility.

Canis familiaris, is a single species divided into more than 400 phenotypically divergent genetic isolates termed breeds, 152 of which are recognized by the American Kennel Club in the United States. Distinct breeds of dog are characterized by ranges of morphology, behaviour, and disease susceptibility.

Different diseases are known to segregate within different purebred dog populations due to inbreeding programs used to generate specific morphologies. Methods for identification of dog breeds may be useful for certifying dogs as belonging to a particular breed. In addition, in the case of a mixed-breed dog, the ability to identify the contribution of different breeds to the mixed-dog's genome, and the specific characteristics of those contributions, may be useful for determining the possible characteristics (e.g. disease susceptibility) of the mixed-breed dog. Existing methods to predict breeds are based on genetics, wherein each breed is defined by a set of single nucleotide polymorphisms or short tandem repeats (STRs), for example.

However, there is a need for further methods of determining the contribution of a dog breed to a test dog genome.

### SUMMARY OF THE INVENTION

In a first aspect, the present invention provides a method of determining the contribution of a dog breed to a test dog genome, comprising:
a) providing a DNA methylation profile from a sample obtained from the test dog; and
b) determining the contribution of a dog breed to the test dog genome by comparing at least part of the DNA methylation profile of the test dog to reference DNA methylation profiles from different dog breeds.

The present invention also provides a method of determining the contribution of a dog breed to a test dog genome, comprising:
a) providing a DNA methylation profile from a sample obtained from the test dog; and
b) determining the contribution of a dog breed to the test dog genome by comparing at least part of the DNA methylation profile of the test dog to a reference DNA methylation profile from at least one reference dog breed.

Suitably, assessing a dog's breed may allow a diet, drug or lifestyle to be selected which contributes to improving the health and wellness of the dog. For example, the assessment may be based on using the size of the breed determined as contributing to the test dog genome, to recommend a diet suitable for e.g. the size of the dog breed. Another example is based on breed body type, as some breeds may be classified as athletic or robust breeds. The breed defined as contributing to the test dog genome may be a pure-breed (e.g. as defined by the American Kennel Club), or a clade or a cluster (e.g. as defined by a phylogenic breed wheel—e.g. Parker et at.; Cell Reports; 2017; 19, 697-708).

Assessing the dog's breed make up may be particularly useful for mixed-breed dogs. For example, the epigenetic portion of each breed in the mixed-breed dog may be used to specifically assess which of the pure breed characteristics was passed on to the mixed breed dog. This determination may then be used to specify a diet, drug or lifestyle to improve the health and wellness of the mixed-breed dog.

For example, many pure breeds have predispositions to particular diseases or conditions. For example, Afghan hounds are predisposed to glaucoma, hepatitis, and hypothyroidism; Basenji are predisposed to coliform enteritis and pyruvate kinase deficiency; Beagles are predisposed to bladder cancer and deafness; Bernese Mountain dogs are predisposed to cerebellar degeneration; Border Terriers are predisposed to oligodendroglioma; and Labrador Retrievers are predisposed to food allergies. Of the genetic diseases discovered in dogs, 46% are believed to occur predominantly or exclusively in one or a few breeds (Patterson et al. (1988) J Am. Vet. Med. Assoc. 193:1131.) Therefore, information regarding the contributions of one or more breeds to the genome of the test genome is particularly valuable to mixed-breed canid owners or caretakers for the purpose of proactively considering health risks for individual tested animals. For example, a mixed breed dog that is found to be a mixture of Newfoundland and Bernese Mountain Dog may be actively monitored for genetic diseases that occur with rare frequency in the general population of dogs, but occur with significant frequency in these specific breeds; thus, a mixed-breed individual of this type would benefit from screens for malignant histiocytosis. Health-related information may also include potential treatments, special diets or products, diagnostic information, and insurance information.

In a further aspect, the invention provides a method for selecting a dietary, pharmacological or lifestyle regime for a test dog, the method comprising:
a) providing a DNA methylation profile from a sample obtained from the test dog;
b) determining the contribution of a dog breed to the test dog genome by comparing at least part of the DNA methylation profile of the test dog to reference DNA methylation profiles from different dog breeds; and
c) selecting a suitable dietary, pharmacological or lifestyle regime for the test dog based on the contribution of a dog breed to the test dog genome determined in step b).

The invention further provides a method for preventing or reducing the risk of a test dog developing a disease; the method comprising:
a) providing a DNA methylation profile from a sample obtained from the test dog;
b) determining the contribution of a dog breed to the test dog genome by comparing at least part of the DNA methylation profile of the test dog to reference DNA methylation profiles from different dog breeds; wherein at least one dog breed contributing to the test dog genome is associated with a propensity to develop a disease; and
c) selecting a dietary, pharmacological or lifestyle regime for the test dog based on the contribution of the at least one dog breed to the test dog genome determined in step b); wherein the pharmacological, lifestyle or dietary regime prevents or reduces the risk of the test dog developing the disease.

Accordingly, the present invention enables a suitable dietary, pharmacological or lifestyle regime to be selected for the dog, based on the contribution of a dog breed to the test dog genome as determined from the DNA methylation profile.

As used herein, ‘selecting a suitable dietary, pharmacological or lifestyle regime for a dog’ may also encompass ‘recommending a dietary, pharmacological or lifestyle regime for the dog’ or ‘providing a recommended dietary, pharmacological or lifestyle regime for the dog’.

The disease may be associated with a morbidity or predicted morbidity of (i) a tissue; (ii) an organ; or (iii) a physiological system, such as the immune, gastrointestinal, urinary, muscular, cardiovascular, and/or neurological system.

The disease may be osteoarthritis, dementia, cognitive dysfunction, pre-diabetic condition, diabetes, cancer, heart disease, obesity, gastrointestinal disorders, incontinence, kidney disease, sarcopenia, vision loss, hearing loss, osteoporosis, cataracts, cerebrovascular disease, and/or liver disease.

Suitably, the disease is a breed-related disease. For example, the breed-related disease may be osteoarthritis, dementia, cognitive dysfunction, pre-diabetic condition, diabetes, cancer, heart disease, obesity, gastrointestinal disorders, incontinence, kidney disease, sarcopenia, vision loss, hearing loss, osteoporosis, cataracts, cerebrovascular disease, and/or liver disease.

The method may optionally further comprise administering the dietary, pharmacological or lifestyle regime to the dog.

The lifestyle or dietary regime may be a dietary intervention. The dietary intervention may be a calorie-restricted diet, a senior diet or a low protein diet.

The invention further provides a dietary intervention for use in preventing or treating a disease in a dog, wherein the dietary intervention is administered to a dog with a breed contribution determined by the method of the invention.

The invention further provides a computer-readable medium comprising instructions that when executed cause one or more processors to perform the method of the invention.

The invention also provides a computer system for selecting a dietary, pharmacological or lifestyle regime for a test dog, the computer system programmed to perform the steps of:
a) determining the contributions of a dog breed to the test dog genome by comparing at least part of a DNA methylation profile obtained from the test dog to reference DNA methylation profiles from different dog breeds; and
b) selecting a suitable pharmacological, lifestyle or dietary regime for the test dog based on the contribution of the dog breed to the test dog genome determined in step a).

In another aspect, the invention provides a computer program product comprising computer implementable instructions for causing a programmable computer to determine the contributions of a dog breed to a test dog genome by comparing at least part of a DNA methylation profile obtained from a test dog to reference DNA methylation profiles from different dog breeds.

In a further aspect the invention provides a computer program product comprising computer implementable instructions for causing a programmable computer to select a dietary, pharmacological or lifestyle regime for a test dog by a) determining the contributions of a dog breed to the test dog genome by comparing at least part of a DNA methylation profile obtained from the test dog to reference DNA methylation profiles from different dog breeds; and b) selecting a suitable pharmacological, lifestyle or dietary regime for the test dog based on the contributions of the dog breed to the test dog genome determined in step a).

### SUMMARY OF THE FIGURES

FIG. 1—A UMAP of all samples using all completely observed methylation sites illustrates that methylation can accurately classify Beagle from Labrador. A binomial LASSO classifier trained on ⅔ of the data is able to accurately classify the 2 breeds using 19 methylation sites.

FIG. 2—Examples of breeds categorised as robust or athletic

FIG. 3—Illustrative breed clades as classified by Parker et al. (Cell Reports; 2017; 19, 697-708).

FIG. 4—T-SNE of the 200 selected methylation sites used in the classifier. The darker colour indicates misclassified dogs while the lighter colour indicates correctly classified dogs, the shape indicates the true breed. Predictions were determined using a SVM classifier fitted on all data except one dog and the model was used to predict the hold out dog breed. Dogs appearing as outliers on the above T-SNE were left out of the training set.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by veterinarians, health-care professionals, lab technicians, pet care providers and so on.

Subject

The present methods are directed to canine subjects. Accordingly, the subject of the present invention is a dog.

Breed

The present invention relates to a method for determining the contribution of a dog breed to a test dog genome using a DNA methylation profile. In particular, the invention provides a method of determining the contribution of a dog breed to a test dog genome, comprising: a) providing a DNA methylation profile from a sample obtained from the test dog; and b) determining the contribution of a dog breed to the test dog genome by comparing at least part of the DNA methylation profile of the test dog to reference DNA methylation profiles from different dog breeds.

Step b) of the method may comprise determining the contribution of a dog breed to the test dog genome by comparing at least part of the DNA methylation profile of the test dog to a reference DNA methylation profile from at least one reference dog breed.

Accordingly, the present invention may be used to determine the breed of a dog or the probability it belongs to a given breed.

Suitably, the present methods may be used to determine the contribution of one or more dog breeds to a test dog genome.

Suitably, the test dog genome is from a mixed-breed dog and the present methods may be used to determine the contribution of one or more dog breeds to the mixed-breed dog genome.

The present methods may be used to determine the contribution of at least 1, at least 2, at least 3, at least 4, at least 5, at least 6, at least 7, at least 8, at least 9, at least 10, at least 20, at least 30, at least 50, at least 100, at least 150, at least 200, at least 300 or at least 400 dog breeds to the test dog genome.

The present methods may be used to determine the contribution of at least 1, at least 2, at least 3, at least 4, at least 5, at least 6, at least 7, at least 8, at least 9, at least 10, at least 20, at least 30, at least 50, at least 100, at least 150, or at least 200 dog breeds to the test dog genome.

The present methods may be used to determine the contribution of about 1 to about 400, about 1 to about 300, about 1 to about 200, about 1 to about 100, about 1 to about 50, about 1 to about 20 or about 1 to about 10 dog breeds to the test dog genome. The present methods may be used to determine the contribution of about 3 to about 400, about 3 to about 300, about 3 to about 200, about 3 to about 100, about 3 to about 50, about 3 to about 20 or about 3 to about 10 dog breeds to the test dog genome. The present methods may be used to determine the contribution of about 5 to about 400, about 5 to about 300, about 5 to about 200, about 5 to about 100, about 5 to about 50, about 5 to about 20 or about 5 to about 10 dog breeds to the test dog genome.

The present methods may be used to determine the contribution of at least 1, at least 2, at least 3, at least 4, at least 5, at least 6, at least 7, at least 8, at least 9 or at least 10 dog breeds to the test dog genome.

Suitably the present methods may be used to determine the contribution of at least one, at least two, or at least three dog breeds to the test dog genome.

Suitably the present methods may be used to determine the contribution of one, two or three dog breeds to the test dog genome.

Suitably the present methods may be used to determine the contribution of two dog breeds to the test dog genome.

Suitably the present methods may be used to determine the contribution of one dog breed to the test dog genome.

The dog breed may be a pure breed, for example a breed as defined by the American Kennel Club (http://www.akc.org/). Examples of pure breeds include, but are not limited to, Afghan Hound, Airedale Terrier, Akita, Alaskan Malamute, American Eskimo Dog, American Foxhound, American Hairless Rat Terrier, American Staffordshire Terrier, American Water Spaniel, Australian Cattle Dog, Australian Shepherd, Australian Terrier, Basenji, Basset Hound, Beagle, Bearded Collie, Bedlington Terrier, Belgian Laekenois, Belgian Malinois, Belgian Sheepdog, Belgian Tervuren, Bernese Mountain Dog, Bichon Frise, Bloodhound, Border Collie, Border Terrier, Borzoi, Boston Terrier, Bouvier des Flandres, Boykin Spaniel, Boxer, Briard, Brittany, Bulldog, Brussels Griffon, Bullmastiff, Bull Terrier, Caim Terrier, Cardigan Welsh Corgi, Cavalier King Charles Spaniel, Chesapeake Bay Retriever, Chihuahua, Chinese Crested, Chinese Shar-Pei, Chow Chow, Clumber Spaniel, Cocker Spaniel, Collie, Curly-Coated Retriever, Dachshund, Dalmatian, Dandie Dinmont Terrier, Doberman Pinscher, Dogo Canario, English Cocker Spaniel, English Foxhound, English Setter, English Springer Spaniel, Entlebucher Mountain Dog, Field Spaniel, Flat-Coated Retriever, French Bulldog, German Longhaired Pointer, German Shepherd Dog, German Shorthaired Pointer, German Wirehaired Pointer, Giant Schnauzer, Golden Retriever, Gordon Setter, Great Dane, Great Pyrenees, Greater Swiss Mountain Dog, Greyhound, Harrier, Havanese, Ibizan Hound, Irish Setter, Irish Terrier, Irish Water Spaniel, Irish Wolfhound, Italian Greyhound, Jack Russell Terrier, Keeshond, Kerry Blue Terrier, Komondor, Kuvasz, Labrador Retriever, Leonberger, Lhasa Apso, Lowchen, Maltese, Manchester Terrier-Standard, Manchester Terrier-Toy, Mastiff, Miniature Bull Terrier, Miniature Pinscher, Miniature Poodle, Miniature Schnauzer, Munsterlander, Neapolitan Mastiff, Newfoundland, New Guinea Singing Dog, Norwegian Elkhound, Norwich Terrier, Old English Sheepdog, Papillon, Pekingese, Pembroke Welsh Corgi, Petit Basset Griffon Vendeen, Pharaoh Hound, Pointer, Polish Lowland Sheepdog, Pomeranian, Portuguese Water Dog, Presa Canario, Pug, Puli, Pumi, Rhodesian Ridgeback, Rottweiler, Saint Bernard, Saluki, Samoyed, Schipperke, Scottish Deerhound, Scottish Terrier, Silky Terrier, Shetland Sheepdog, Shiba Inu, Shih Tzu, Siberian Husky, Smooth Fox Terrier, Soft Coated Wheaten Terrier, Spinone Italiano, Staffordshire Bull Terrier, Standard Poodle, Standard Schnauzer, Sussex Spaniel, Tibetan Spaniel, Tibetan Terrier, Toy Fox Terrier, Toy Poodle, Vizsla, Weimaraner, Welsh Springer Spaniel, Welsh Terrier, West Highland White Terrier, Wirehaired Pointing Griffon, Whippet, and Yorkshire Terrier.

Suitably, the dog breed may refer to a group or clade of pure breeds categorised based on one or more criteria.

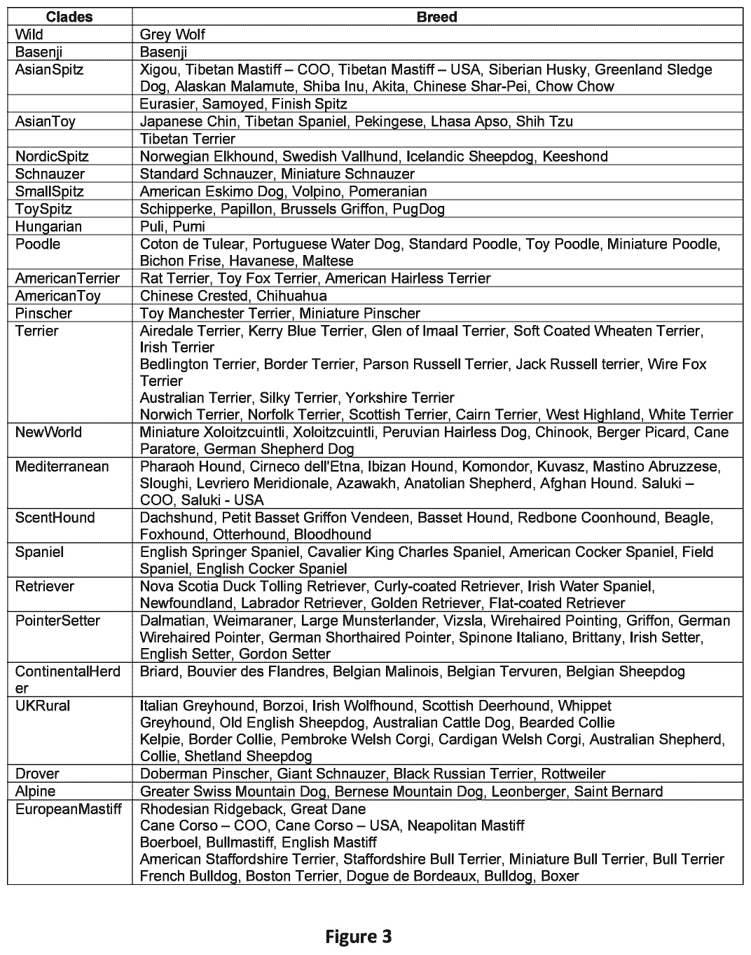

Breeds may be categorised into clades based on genetic distance, optionally in combination with additional factors such as migration, and genome-wide haplotype sharing analyses. An example of breed clade categorisation is described in Parker et al. (Cell Reports; 2017; 19, 697-708), in which 161 dog breeds analysis were classified into 23 breed clades. Illustrative breed clades as classified by Parker et al. are shown in FIG. 3.

Suitably, the breed clade may be selected from Wild, Basenji, Asian Spitz, Asian Toy, Nordic Spitz, Schnauzer, Small Spitz, Toy Spitz, Hungarian, Poodle, American Terrier, American Toy, Pinscher, Terrier, New World, Mediterranean, Scent Hound, Retriever, Pointer Setter, Continental Herder, UK Rural, Drover, Alpine, and European Mastiff. The breed clades may comprise the pure breeds shown in FIG. 3, for example.

The breeds may be classified based on the size of the breed, for example based on the average size of the breed. For example, the breed may be categorised as a toy, small, medium, large or giant breed. Suitably, the dog breed may be categorised based on the weight of the dog. Suitably, the dog breed may be categorised based on the average weight of a dog for a given breed. A “miniature breed” may refer to a breed with an average weight of less than 5 kg. A “small breed” may refer to a breed with an average weight between 5 and 10 kg. A “medium breed” may refer to a breed with an average weight between 10 and 25 kg. A “large breed” may refer to a breed with an average weight between 25 and 40 kg. A “giant breed” may refer to a breed with an average weight of more than 40 kg.

Suitably, the breeds may be classified based on body conformation types. For example, the breed may be classified as robust or athletic. Certain breeds may be grouped as robust or athletic using methods such as those described in EP1983842. Suitably, body type conformations are influenced by and dependent upon a variety of factors, including the body mass index, body composition, daily energy requirement, resting metabolic rate, dog breed, and genetics differentiation during breeding history.

The body mass index may be calculated by the following formula: weight (kg)/[shoulder height (m)]². A robust dog will generally have a body mass index greater than 90 kg/m². An athletic dog will generally have a body mass index less than 90 kg/m². Examples of typical BMI values for robust dogs or athletic dogs are:

| Robust dogs | Athletic dogs |
|---|---|
| Saint Bernard: 158.2 kg/m² | Greyhound: 54.8 kg/m² |
| Bull dog: 211.5 kg/m² | Irish setter: 61.6 kg/m² |
| Pekingese: 99.6 kg/m² | Fox terrier: 51.8 kg/m² |

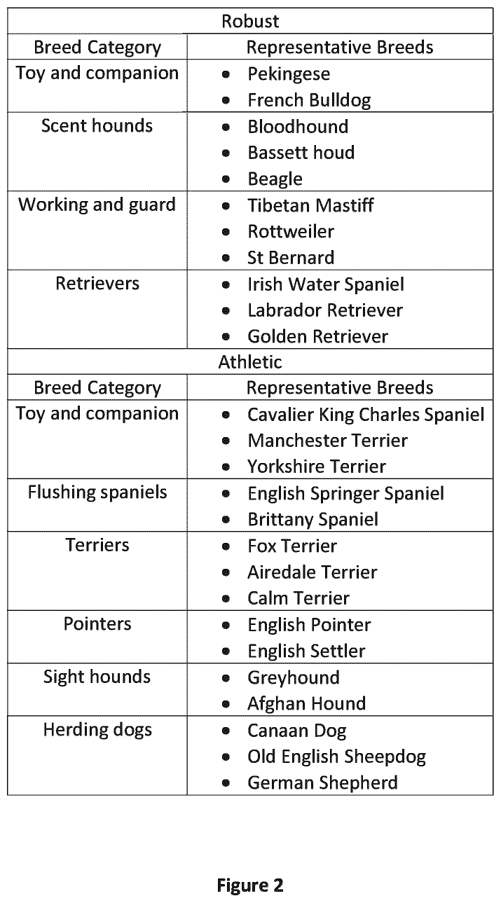

Examples of breeds categorised as robust or athletic are shown in FIG. 2.

Categorizing a dog as robust or athletic may also be influenced by the breeding history of a dog. For instance, dogs may have a different breeding history and genetic background than the breed category in which they are primarily categorized. Generally, dogs having some athletic blood in their breeding history tend to have kept the athletic morphology as a dominant phenotype and have higher energy needs. For example, the Great Dane that belongs to the working and guard dog group (and therefore should be classified as a robust dog) may be classified as athletic because of its morphology and breeding history (sight hounds blood). It has a clear athletic type body conformation, i.e., deep chest and thin abdomen and high daily energy requirements to maintain his ideal body weight.

Assessing the dog's breed make up may be particularly useful for mixed-breed dogs. In particular, the methylation profile may be used to determine the contribution of different breeds to the test dog's genome. For example, the methylation profile may be used to determine the percentage contribution of different dog breeds to the test dog's genome. The methylation profile may also be used to identify the regions of the test dog genome which are similar to, or have been inherited from, a given breed. This may be particularly advantageous in circumstances where a given genomic region or locus is known to co-segregate with, for example, disease susceptibility or particularly behavioural characteristics.

Sex

Suitably, the sex of the dog may be classified as male or female. Suitably, the sex of the dog may be included in the present methods (e.g. in a regression analysis as described herein).

Chronological Age

Chronological age may be defined as the amount of time that has passed from the subject's birth to the given date. Chronological age may be expressed in terms of years, months, days, etc.

Suitably, the present method may be applied to a dog of any chronological age.

Suitably, the chronological age of the dog may be included in the present methods (e.g. in a regression analysis as described herein).

Biological Age

Depending on genetics, nutrition, and lifestyles—for example—individuals may age slower or faster than their chronological age. Chronological age may therefore not always reflect an individual's biological rate of aging. As such, the biological age of an individual (based on e.g. clinical biochemistry and cell biology measures) can vary compared to others of the same chronological age. Methods for determining biological age are known in the art and include, for example, methods utilising methylation profiles, clinical chemistry panel profiles or telomere length.

Suitably, the present method may be applied to a dog of any biological age.

Suitably, the biological age of the dog may be included in the present methods (e.g. in a regression analysis as described herein).

Sample

The present invention comprises a step of providing a DNA methylation profile from one or more samples obtained from a subject.

The present invention comprises a step of determining a DNA methylation profile from one or more samples obtained from a subject.

Suitably, the sample is a blood, hair follicle, buccal swab, saliva, fecal or tissue sample.

Suitably, the sample is derived from blood. The sample may contain a blood fraction or may be whole blood. The sample preferably comprises whole blood. The sample may comprise a peripheral blood mononuclear cell (PBMC) or lymphocyte sample. Techniques for collecting samples from a subject and extracting DNA (e.g. genomic DNA) from the sample are well known in the art.

Suitably, the sample is a hair follicle, buccal swab or saliva sample. Such sample types are particularly applicable if the sample is to be provided, for example, outside of a veterinarian environment—for example using a kit for home use.

DNA Methylation

DNA methylation is the process by which a methyl group (CH₃) is added covalently to a cytosine base that is part of a DNA molecule. In vivo, this process is catalysed by a family of DNA methyltransferases (Dnmts), that generate the modified cytosine by transfer of a methyl group from S-adenyl methionine (SAM). The cytosine is modified on the 5^{th} carbon atom, and the modified residue is known as 5-methylcytosine (5mC). The DNA methylation may also comprise 5-hydroxymethylcytosine (5hmc).

DNA methylation is an example of an epigenetic mechanism, i.e. it is capable of modifying gene expression without modification of the underlying DNA sequence. DNA methylation can, for example, inhibit the expression of genes by acting as a recruitment signal for repressive factors, or by directly blocking transcription factor recruitment. DNA methylation predominantly occurs in the genome of somatic mammalian cells at sites of adjacent cytosine and guanine that form a dinucleotide (CpG). While non-CpG methylation is observed in embryonic development, in the adult these modifications are much reduced in most cell types. CpG islands are stretches of DNA that have a high CpG density, but are generally unmethylated. These regions are associated with promoter regions, particularly promoter regions of housekeeping genes, and are thought to be maintained in a permissive state to allow gene expression.

The detection of specific methylated DNA can be accomplished by multiple methods (see e.g. Zuo et al., 2009; Epigenomics. 1(2):331-345) and Rauluseviciute et al.; Clinical Epigenetics; 2019; 11(193)). A number of methods are available for detection of differentially methylated DNA at specific loci in samples such as blood, urine, stool or saliva. These methods are able to distinguish 5-methyl cytosine or methylated DNA from unmethylated DNA, and subsequently quantify the proportion of methylated and unmethylated DNA for a particular genomic site.

The present methods may comprise determining a DNA methylation profile for dog using any suitable method. Suitable methods include, but are not limited to, those described below.

Enzymatic Methyl-Seq (EM-Seq)

Suitably, enzymatic approaches are used to detect 5mC and 5hmC. By way of example, Enzymatic Methyl-seq (EM-seq) may be used.

Typically in EM-seq, in a first enzymatic step, 5mC is oxidized to 5hmC, then 5fC and finally 5caC by the activity of Tet methylcytosine dioxygenase 2 (TET2). In addition, use of a T4-BGT enzyme glucosylates both the pre-existing 5hmC and that produced by TET2 activity. In a second enzymatic step, following denaturation of the double-stranded DNA, the enzyme apolipoprotein B mRNA editing enzyme catalytic polypeptide-like 3A (APOBEC3A) is used to deaminate cytosines, but is unable to deaminate the oxidised or glycosylated forms of 5mC and 5hmC. Only unmethylated cytosines are deaminated to form uracil bases. Prior to the first enzymatic step, the DNA fragments may be generated from mechanical shearing and end-repaired, A-tailed, and ligated to sequencing adaptors, which can be carried out using the NEBNext® DNA Ultra II reagents (NEB), for example. Following the second enzymatic step, the deaminated single-stranded DNA may be amplified by PCR reactions, using polymerase such as NEBNext® Q5M which can amplify uracil containing templates, and the resulting library can be sequenced or analysed in an identical manner to the DNA sample generated by bisulfite sequencing. The output of EM-seq is generally the same as whole genome bisulfite sequencing, but with the use of less DNA-damaging reagents, which consequently reduces sample loss, and can outperform bisulfite-conversion prepared samples in coverage, sensitivity and accuracy of cytosine methylation calling. An illustrative EM-seq method is described by Vaisvila et al. (Genome Research; 2021; 31:1-10).

Bisulfite Conversion-Based Methods

Bisulfite conversion utilizes the selective conversion of unmethylated cytosines to uracil when treated with sodium bisulfite. Denatured DNA is treated with sodium bisulfite, which converts all unmodified cytosines to uracil, and subsequent PCR amplification converts these residues to thymines. Analysing the produced DNA sequences can be done via many different methods, examples of which include but are not limited to: denaturing gel electrophoresis, single-strand conformation polymorphism, melting curves, fluorescent real-time PCR (MethyLight), MALDI mass spectrometry, array hybridization, and sequencing (e.g. Whole Genome Bisulfite Sequencing WGBS). Recently developed techniques such as SeqCap Epi enrich sequences of interest prior to sequencing that enables deeper coverage over a more focused area). Comparison of the abundance of sequences in a bisulfite-converted sample against those of an untreated control allows analysis of methylation at a target site, where the proportion of converted sequences is indicative of the level of methylation at the target site.

Further variants of the bisulfite conversion method are available that are able to distinguish 5mC from the oxidised form 5-hydroxymethylcytosine (5hmC), which behaves identically to 5mC under standard bisulfite conversion, and to detect the further modification 5-formylcytosine (5fC). These methods, such as oxBS-Seq and redBS-Seq, utilise oxidation and reduction of these markers to modify the susceptibility of each species to bisulfite conversion, and through comparative analysis quantify the amount of each modification at target loci.

Selective Restriction Endonuclease Digestion Methods

Methods of analysing DNA methylation patterns exist may involve the use of restriction enzymes. These include, for example, restriction landmark genomic scanning (RLGS) (Costello et al., 2000; Nat Genet.;24(2):132-8), methylation-sensitive representational difference analysis (MS-RDA) (Ushijima et al., Proc Natl Acad Sci USA. 1997 Mar. 18; 94(6):2284-9), and differential methylation hybridization (DMH) (Huang et al., Cancer Res. 1997 Mar. 15; 57(6):1030-4). Restriction endonucleases can be methylation dependent in their digestion activity. This specificity can be used to differentiate methylated and unmethylated sequences. Certain restriction enzymes, for example BstUl, Hpall and Notl are sensitive to methylated recognition sequences. Others, such as McrBC, are specific for methylated sequences.

As an example, differential methylation hybridisation (DMH) (Huang et al., as above]) requires an initial fragmentation of the genome with a bulk genome restriction enzyme, such as Msel, which fragments the genome into lengths of less than 200 bp. Following this step, the genome fragments are digested using a methylation-sensitive restriction endonuclease (MREs), or in some versions of the technique, a cocktail of MREs to improve coverage. Depending on the specificity of enzyme or enzymes used, either the methylated or the unmethylated sequences will be degraded.

Digested sequences will not be amplified in a subsequent PCR step. The resultant PCR products are suitable for further processing and analysis by sequencing or microarray hybridisation in combination with fluorescent dyes.

Suitably, the present methods utilise a DNA methylation profile generating by a method comprising the use of one or more MREs.

Suitable comparators can be used to investigate methylation state between conditions. DNA from healthy subjects can be compared with aged or diseased subjects to detect changes in methylation state (Huang et al., Hum Mol Genet. 1999 Mar.;8(3):459-70). Alternatively, a methylation-insensitive version of the secondary digest enzyme, such as the HpalI isoschizomer Mspl, can be used to generate a control sample, so that intra- or inter- genomic DNA methylation comparisons can be made (Khulan et al., Genome Res. 2006 August; 16(8):1046-55).

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. Alternatively, the method can encompass amplifying intact DNA after restriction enzyme digestion, thereby only amplifying DNA that was not cleaved by the restriction enzyme in the area amplified. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

Suitably, the digestion of nucleic acid is detected by selective hybridization of a probe or primer to the undigested nucleic acid. Alternatively, the probe selectively hybridizes to both digested and undigested nucleic acid but facilitates differentiation between both forms, e.g., by electrophoresis. Suitable detection methods for achieving selective hybridization to a hybridization probe include, for example, Southern or other nucleic acid hybridization.

Suitable hybridization conditions may be determined based on the melting temperature (Tm) of a nucleic acid duplex comprising the probe. The skilled artisan will be aware that optimum hybridization reaction conditions should be determined empirically for each probe, although some generalities can be applied. Preferably, hybridizations employing short oligonucleotide probes are performed at low to medium stringency. In the case of a GC rich probe or primer or a longer probe or primer a high stringency hybridization and/or wash is preferred. A high stringency is defined herein as being a hybridization and/or wash carried out in about 0.1×SSC buffer and/or about 0.1% (w/v) SDS, or lower salt concentration, and/or at a temperature of at least 65° C., or equivalent conditions. Reference herein to a particular level of stringency encompasses equivalent conditions using wash/hybridization solutions other than SSC known to those skilled in the art.

Reduced Representation Bisulfite Sequencing (RRBS)

Reduced representation bisulfite sequencing (RRBS) enriches CpG-rich genomic regions using the Mspl restriction enzyme—which cuts DNA at all CCGG sites, regardless of their DNA methylation status at the CG site—and enables the measurement of DNA methylation levels at 5%˜ 10% of all CpG sites in the mammalian genome.

As such, the method involves digestion of DNA using the methylation-insensitive Mspl prior the bisulfite conversion and sequencing. Using Mspl to digest genomic DNA results in fragments that always start with a C (if the cytosine is methylated) or a T (if a cytosine was not methylated and was converted to a uracil in the bisulfite conversion reaction). This results in a non-random base pair composition. Additionally, the base composition is skewed due to the biased frequencies of C and T within the samples. Various software for alignment and analysis is available, such as Maq, BS Seeker, Bismark or BSMAP. Alignment to a reference genome allows the programs to identify base pairs within the genome that are methylated.

Affinity Enrichment Based Methods

Distinction of methylated from unmethylated DNA can be accomplished by the use of antibodies, such as anti-5mC, and/or methylated-CpG binding proteins, that contain a methyl-CpG-binding domain (MBD). The antibodies of MBD-domain proteins are able to specifically isolate methylated DNA over unmethylated DNA. Methods that utilize antibodies are commonly referred to as MeDIP, whilst methods utilizing methylated-CpG binding proteins are often known as MBD or MIRA approaches.

These methods require initial fragmentation of the genome, which can be carried out with bulk genome digest with an enzyme such as Msel, which cuts frequently, followed by affinity purification of methylated fragments. The input DNA can be compared to the purified methylated DNA by microarray hybridisation or sequencing to obtain comparative analysis of methylation levels at specific sites.

Further variants of affinity enrichment-based methods are available, such as MethylCap-Seq or MBD-Seq. These methods reduce sample complexity by using a salt gradient to elute methylated DNA fragments in a methy-CpG-abundance dependent manner, segregating CpG islands and other highly methylated loci from less CpG dense loci. The fractions can then be sequenced separately improving sequence coverage.

Single Molecule Sequencing-Based and De Novo Methylation Sequencing Approaches

Contemporary sequencing methods are able to sequence single molecules directly. Single-molecule real-time (SMRT) DNA sequencing is available, for example the Sequel systems from Pacific Biosciences and has been shown to be able to identify modified bases such as methylated cytosine based on the polymerase kinetics. Nanopore sequencing devices, such as the MinION nanopore sequencer from Oxford Nanopore Technologies, which are able to individually sequence long strands of DNA, are also able to detect de novo base modifications, including methylation.

DNA Methylation Sites

Suitably, a DNA methylation site may refer to the presence or absence of a 5mC at a single cytosine, suitably a single CpG dinucleotide.

Suitably, a DNA methylation site may refer to the presence or absence of methylation (i.e. the number of 5mC or percentage of 5mC) across a plurality of CpG sites within a DNA region. Suitably, a DNA methylation site may refer to the level of methylation (i.e. the number of 5mC or percentage of 5mC) across a plurality of CpG sites within a DNA region. A “DNA region” may refer to a specific section of genomic DNA. These DNA regions may be specified either by reference to a gene name or a set of chromosomal coordinates. Both the gene names and the chromosomal coordinates would be well known to, and understood by, the person of skill in the art.

Suitably, gene names and/or coordinates may be based on the “Tasha” dog reference genome (https://www.ncbi.nlm.nih.gov/assembly/GCF_000002285.5; Jagannathan et al.; Genes (Basel); 2021; 12(6); 847).

The DNA region may define a section of DNA in proximity to the promoter of a gene, for example. Promoter regions are known to be rich in CpG. By way of example, the DNA region may refer to about 3kb upstream to about 3kb downstream; about 2kb upstream to about 2kb downstream; about 2kb upstream to about 1kb downstream; about 2kb upstream to about 0.5kb downstream; about 1kb upstream to about 0.5kb downstream; about 0.5kb upstream to about 0.5kb downstream of a promoter. Suitably, the DNA region may refer to about 1kb upstream to about 0.5kb downstream of a promoter.

Suitably, the DNA region may comprise or consist of CpG sites that are less than about 5000, less than about 4000, less than about 3000, less than about 2000, less than about 1000, less than about 500, or less than about 200 bases apart.

Suitably, the DNA region may comprise or consist of CpG sites that are between about 200 to about 5000, about 200 to about 4000, about 200 to about 3000, about 200 to about 2000, or about 200 to about 1000 bases apart.

Suitably, the DNA region may comprise one or more CpG islands. Suitably, the DNA region may consist of a CpG island.

A “CpG island” may refer to a DNA region comprising at least 200 bp, a GC percentage greater than 50%, and an observed-to-expected CpG ratio greater than 60%.

Suitably, the DNA methylation sites do not comprise X and/or Y chromosome CpGs.

Suitably, the DNA methylation sites do not comprise CpGs known to comprise a SNP at the CpG.

Reference to each of the genes/DNA regions detailed above should be understood as a reference to all forms of these molecules and to fragments or variants thereof. As would be appreciated by the person of skill in the art, some genes are known to exhibit allelic variation between individuals or single nucleotide polymorphisms. Variants include nucleic acid sequences from the same region sharing at least 90%, 95%, 98%, 99% sequence identity i.e. having one or more deletions, additions, substitutions, inverted sequences etc. relative to the DNA regions described herein.

Accordingly, the present invention should be understood to extend to such variants which, in terms of the present applications, achieve the same outcome despite the fact that minor genetic variations between the actual nucleic acid sequences may exist between individuals. The present invention should therefore be understood to extend to all forms of DNA which arise from any other mutation, polymorphic or allelic variation.

In terms of screening for the methylation of these gene regions, it should be understood that the assays can be designed to screen for specific DNA. It is well within the skill of the person in the art to choose which strand to analyse and to target that strand based on the chromosomal coordinates. In some circumstances, assays may be established to screen both strands.

“Methylation status” may be understood as a reference to the presence, absence and/or quantity of methylation at a particular nucleotide, or nucleotides, within a DNA region. The methylation status of a particular DNA sequence (e.g. DNA region as described herein) can indicate the methylation state of every base in the sequence or can indicate the methylation state of a subset of the base pairs (e.g, of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.”

Suitably, DNA methylation may be determined using an EM-Seq strategy. In such methods, a methylation level can be determined as the fraction of ‘C’ bases out of ‘C’+‘U’ total bases at a target CpG site “i” following an enzyme and APOBEC3A conversion treatment. In other embodiments, the methylation level can be determined as the fraction of ‘C’ bases out of ‘C’+‘T’ total bases at site “i” following enzyme and APOBEC3A conversion treatment and subsequent nucleic acid amplification. The mean methylation level at each site may then be evaluated to determine if one or more threshold is met.

In some embodiments, in particular when bisulfite conversion and sequencing methods are used, a methylation level can be determined as the fraction of ‘C’ bases out of ‘C’+‘U’ total bases at a target CpG site “i” following a bisulfite treatment. In other embodiments, the methylation level can be determined as the fraction of ‘C’ bases out of ‘C’+T total bases at site “i” following a bisulfite treatment and subsequent nucleic acid amplification. The mean methylation level at each site may then be evaluated to determine if one or more threshold is met.

Alternatively, a methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of the methylation status. This is of particular use when it is desirable to compare the methylation status of a sequence in a sample to a threshold value.

The present invention is not to be limited by a precise number of methylated residues that are considered to indicative of breed, because some variation between samples will occur. The present invention is also not necessarily limited by positioning of the methylated residue.

In one embodiment, a screening method can be employed which is specifically directed to assessing the methylation status of one or more specific cytosine residues or the corresponding cytosine at position n+1 on the opposite DNA strand.

DNA Methylation Profile

A “DNA methylation profile” or “methylation profile” may refer to the presence, absence, quantity or level of 5mC at one or more DNA methylation sites. Preferably, “methylation profile” refers to the presence, absence, quantity or level of 5mC at a plurality of DNA methylation sites. Thus, the presence, absence, quantity or level of 5mC at each individual DNA methylation site within the plurality of sites may be assessed and contribute to the determination of breed make-up of the dog. The quality and/or the power of the methods may thus be improved by combining values from multiple DNA methylation markers.

Suitably, the present breed profile comprises the methylation profile from a plurality of methylation sites.

Suitably, presence or absence of 5mC from at least 5, at least 10, at least 20, at least 50, at least 100, at least 200, at least 500, at least 1000, at least 2000, at least 5000, at least 10000, at least 50000, at least 10000, at least 250000, or at least 500000 DNA methylation sites may be used to determine the breed profile of the dog.

Suitably, the methylation profile may refer to the presence or absence of 5mC from at least 100, at least 200, at least 500, at least 1000 or at least 2000 DNA methylation sites.

Suitably, the methylation profile may refer to the presence or absence of 5mC from at least least 5, at least 10, at least 20, at least 50, at least 100, or at least 200 methylation sites.

Suitably, the methylation profile may refer to the presence or absence of 5mC from about 5, about 10, about 20, about 50, about 100, about 200, about 500, about 1000 or about 2000 DNA methylation sites.

Suitably, the methylation profile may refer to the presence or absence of 5mC from about 100, about 200, about 500, about 1000 or about 2000 DNA methylation sites.

Suitably, the methylation profile may refer to the presence or absence of 5mC from about 5, about 10, about 20, about 50, about 100, or about 200DNA methylation sites.

In order to generate a breed profile, an initial methylation profile may be processed or streamlined to produce a restricted methylation profile which is then used to generate the bred profile.

By way of example, an initial methylation profile may be processed or streamlined by—for example—using DNA regions rather than individual cytosines, by selecting a subset of methylation sites that are associated with a particular physiological or biochemical pathway, performing a correlation analysis and retaining one or more representative DNA methylation sites per cluster, or performing differential analysis to pre-select DNA methylation sites or retain DNA methylation sites that vary more between different breeds. The processing of the DNA methylation sites may comprise calculating the mutual information for each of the sites, ordering them according to this metric and then selecting the top-n to put in the model.

For example, the DNA region(s) may be any DNA region(s) as defined herein.

Suitably, the methylation profile may refer to DNA methylation sites of genes that are associated with a particular physiological or biochemical pathway. As such, the methylation profile may comprise methylation sites associated with a particular tissue, organ, or physiological system. Determining the status methylation sites associated with a particular tissue, organ or physiological system may advantageously allow the method to be utilised in a way which focuses on pathologies and diseases of that tissue, organ or physiological system. For example, if a particular breed of dog is known to be associated with muscular or cardiovascular disease, it may be advantageous to determine the methylation sites that physiological system.

Suitably, the physiological system may be the immune, gastrointestinal, urinary, muscular, cardiovascular, and/or neurological system.

A methylation profile for a particular tissue, organ, or physiological system may be determined using a DNA methylation profile comprising, or consisting of, methylation sites from genes that are preferentially or specifically expressed by that tissue, organ, or physiological system. Classifications of genes by a particular tissue, organ, or physiological system are publicly available at, for example, Gene Ontology (http://geneontology.org/), the KEGG pathway database (https://www.genome.jp/kegg/), or MSIgDB (https://www.gsea-msigdb.org/gsea/msigdb/index.jsp).

In some embodiments, a threshold selects those sites having the highest-ranked mean methylation values for breed determination. For example, the threshold can be those sites having a mean methylation level that is the top 50%, the top 40%, the top 30%, the top 20%, the top 10%, the top 5%, the top 4%, the top 3%, the top 2%, or the top 1% of mean methylation levels across all sites “i” tested for a predictor, e.g., a breed identification.

Alternatively, the threshold can be those sites having a mean methylation level that is at a percentile rank greater than or equivalent to 50, 60, 70, 80, 90, 95, 96, 97, 98, or 99. In other embodiments, a threshold can be based on the absolute value of the mean methylation level. For instance, the threshold can be those sites having a mean methylation level that is greater than 99%, greater than 98%, greater than 97%, greater than 96%, greater than 95%, greater than 90%, greater than 80%, greater than 70%,greater than 60%, greater than 50%, greater than 40%, greater than 30%, greater than 20%, greater than 10%, greater than 9%, greater than 8%, greater than 7%, greater than 6%, greater than 5%, greater than 4%, greater than 3%, or greater than 2%. The relative and absolute thresholds can be applied to the mean methylation level at each site “i” individually or in combination. As an illustration of a combined threshold application, one may select a subset of sites that are in the top 3% of all sites tested by mean methylation level and also have an absolute mean methylation level of greater than 6%. The result of this selection process is a DNA methylation profile, of specific hypermethylated sites (e.g., CpG sites) that are considered the most informative for breed determination.

Suitably, the DNA methylation profile may comprise at least one methylation site as listed in Table 1.

Suitably, the methylation site(s) may be defined as the methylation markers present in any one or more of SEQ ID NO: 1-200. SEQ ID NO: 1-200 show the sequence either side of the methylation marker in the “Tasha” dog reference genome (https://www.ncbi.nlm.nih.gov/assembly/GCF_000002285.5; Jagannathan et al.; Genes (Bsael); 2021; 12(6); 847). The “CG” methylation marker are the 26^{th} and 27^{th} nucleotides in the sequence (i.e. there are 25 nucleotides preceding the methylation marker and 25 nucleotides following the methylation marker).

Suitably, the methylation site may be defined as the intervening position in the column labelled “Site” in Table 1. For example, for site chr10:10975030-10975032, the methylation marker is chr10: 10975031.

Suitably, the DNA methylation profile may comprise at least 1, at least 2, at least 5, at least 10, at least 20, at least 50, at least 100, at least 150, at least 175 or preferably each of the methylation sites as listed in Table 1.

Suitably, the DNA methylation profile may comprise at least 1, at least 2, at least 5, at least 10, at least 20, at least 50, at least 100, at least 125, or each of the methylation sites listed as ‘Site Number’ 1-150 in Table 1.

Suitably, the DNA methylation profile may comprise at least 1, at least 2, at least 5, at least 10, at least 20, at least 50, at least 75 or each of the methylation sites listed as ‘Site Number’ 1-100 in Table 1.

Suitably, the DNA methylation profile may comprise at least 1, at least 2, at least 5, at least 10, at least 20, at least 30, at least 40 or each of the methylation sites listed as ‘Site Number’ 1-50 in Table 1.

Suitably, the DNA methylation profile may comprise at least 1, at least 2, at least 5, at least 10, at least 15, or each of the methylation sites listed as ‘Site Number’ 1-20 in Table 1.

Suitably, the DNA methylation profile may comprise at least 1, at least 2, at least 3, at least 5 or each of the methylation sites listed as ‘Site Number’ 1-10 in Table 1.

Suitably, the DNA methylation profile may comprise at least 1, at least 2, at least 3 or each of the methylation sites listed as ‘Site Number’ 1-5 in Table 1.

Suitably, the DNA methylation profile may comprise at least one, at least two, at least five, at least ten, at least fifteen or preferably each of the methylation sites as listed in Table 5. This methylation profile is suitable for distinguishing—for example—Beagle and Labrador retriever profiles.

Determination of DNA Methylation Sites/DNA Methylation Profiles Indicative of Breed

The present invention comprises utilising a DNA methylation profile to determine the contribution of a dog breed to a test dog genome. As such, the present invention comprises utilising a DNA methylation profile to determine the contribution of one or more dog breeds to a test dog genome. The contribution of one or more dog breeds to a test dog genome may also be referred to herein as the ‘breed profile’.

The provision of DNA methylation sites or a DNA methylation profile that is indicative of breed may be achieved through training datasets and machine learning approaches, for example. Suitably, the machine learning approaches may be supervised machine learning approaches. Suitably, the model may be a multinomial regression, support-vector machine (SVM) or a random forest model.

By way of example, DNA methylation sites or a DNA methylation profile may be trained against a dataset comprising dogs of a known breed(s). Suitably, the DNA methylation sites or a DNA methylation profile may be trained against a dataset comprising dogs of a known breed(s) in combination with known age and/or sex.

For example, models for DNA methylation sites or a DNA methylation profile indicative of breed contribution may be provided by training a dataset of methylation status at a plurality of DNA methylation sites against a training dataset of dogs with a known breed using a machine learning framework, and testing against a with-held cohort to validate the veracity of the model.

The machine learning framework may comprise fitting a penalized regression to a training dataset of dogs with a known breed (and optionally age and/or sex); for example using glmnet R package.

The machine learning framework may comprise fitting a multinomial model, a random forest, a SVM (support vector machine), penalized multinomial logistic regression or other model used to predict multi class outcomes.

Suitably, the machine learning framework may comprise fitting a penalized regression, such as an elastic net regression, of breed explained by a DNA methylation profile, (and optionally age and/or sex).

Suitably, the machine learning framework may comprise fitting a penalized regression, such as an elastic net regression, of breed explained by a DNA methylation profile, age and sex.

Suitably, the machine learning framework may be used to determine a model comprising a set of DNA methylation sites or a DNA methylation profile that is indicative of breed.

The model may comprise the methylation status at a plurality of DNA methylation sites; wherein the methylation status at each site is considered in the model by multiplying by a coefficient value.

The coefficient value for each parameter typically depends on the measurement units of all the variables in the model. As would be understood by the skilled person, the value for each coefficient value will therefore depend on, for example, the number and nature of the different parameters used in the model and the nature of the training data provided. Accordingly, routine statistical methods may be applied to a training data set in order to arrive at coefficient values.

Suitably, sex may be coded as a numerical value with 0 for female and 1 for male.

Suitably, the machine learning platform may comprise one or more deep neural networks. Neural Networks are collections of neurons (also called units) connected in an acyclic graph. Neural Network models are often organized into distinct layers of neurons. For most neural networks, the most common layer type is the fully-connected layer in which neurons between two adjacent layers are fully pairwise connected, but neurons within a single layer share no connections. One of the main features of deep neural networks is that neurons are controlled by non-linear activation functions. This non-linearity combined with the deep architecture make possible more complex combinations of the input features leading ultimately to a wider understanding of the relationships between them and as a result to a more reliable final output. Deep neural networks have been applied for many types of data ranging from structural data to chemical descriptors or transcriptomics data.

Suitably, the machine learning platform comprises one or generative adversarial networks. Suitably, the machine learning platform comprises an adversarial autoencoder architecture. Suitably, the machine learning platform comprises a feature importance analysis for ranking DNA methylation site by their importance in breed determination.

Comparison to a Reference or Control

The present method may further comprise a step of comparing the difference in DNA methylation at one or more sites in the test sample to one or more reference or controls. The presence or absence of DNA methylation at one or more sites in the reference or control may be associated with a breed. In some embodiments, the reference value is a value obtained previously for a subject or group of subjects with a known breed. The reference value may be based on a known DNA methylation status at one or more sites, e.g. a mean or median level, from a group of subjects with known breed.

The reference DNA methylation profiles may comprise DNA methylation profiles from at least 2, at least 4, at least 10, at least 20, at least 40, at least 80, at least 100, at least 150, at least 200, at least 300 or at least 400 dog breeds.

The reference DNA methylation profiles may comprise DNA methylation profiles from at least 2, at least 4, at least 10, at least 20, at least 40, at least 80, at least 100, at least 150 or at least 200 dog breeds.

Enrichment and Detection Methods

Determining a DNA methylation profile may comprise a step of enriching a DNA sample for selected DNA regions. For example, the methods may comprise a step of enriching a DNA sample for DNA regions comprising the DNA methylation sites which comprise the DNA methylation profile.

Suitable enrichment methods are known in the art and include, for example, amplification or hybridisation-based methods. Amplification enrichment typically refers to e.g. PCR based enrichment using primers against the DNA regions to be enriched. Any suitable amplification format may be used, such as, for example, polymerase chain reaction (PCR), rolling circle amplification (RCA), inverse polymerase chain reaction (iPCR), in situ PCR, strand displacement amplification, or cycling probe technology.

Hybridisation enrichment or capture-based enrichment typically refers to the use of hybridisation probes (or capture probes) that hybridise to DNA regions to be enriched.

The hybridisation probe(s) may be attached directly to a solid support, or may comprise a moiety, e.g. biotin, to allow binding to a solid support suitable for capturing biotin moieties (e.g. beads coated with streptavidin). In any case, DNA comprising sequence which is complementary to the probe may be captured thus allowing to separate DNA comprising DNA regions of interest from not comprising the DNA regions of interest. Hence, such a capturing steps allows to enrich for the DNA regions of interest. For example, the DNA regions may be DNA regions in proximity to gene promoters.

An array used herein can vary depending on the probe composition and desired use of the array. For example, the nucleic acids (or CpG sites) detected in an array can be at least 10, 100, 1,000, 10,000, 0.1 million, 1 million, 10 million, 100 million or more. Alternatively or additionally, the nucleic acids (or CpG sites) detected can be selected to be no more than 100 million, 10 million, 1 million, 0.1 million, 10,000, 1,000, 100 or less. Similar ranges can be achieved using nucleic acid sequencing approaches such as those known in the art; e.g. Next Generation or massively parallel sequencing.

Suitably, an enrichment step may be performed before or after the step of separating or differentially treating methylated and unmethylated DNA.

As used herein, the term “enriching” or “enrichment” for “DNA” or “DNA regions” means a process by which the (absolute) amount and/or proportion of the DNA comprising the desired sequence(s) is increased compared to the amount and/or proportion of DNA comprising the desired sequence(s) in the starting material. In this regard, enrichment by amplification increases the amount and proportion of the desired sequence(s). Enrichment by capture-based enrichment increases the proportion of DNA comprising the desired sequence(s).

Following processing of the DNA to distinguish methylated and unmethylated sites, the present methods may further comprise the step of identifying the sites which were methylated or unmethylated (i.e. in the original sample).

The identification step may comprise any suitable method known in the art, for example array detection or sequencing (e.g. next generation sequencing).

A sequencing identification step preferably comprises next generation sequencing (massively parallel or high throughput sequencing). Next generation sequencing methods are well known in the art, and in principle, any method may be contemplated to be used in the invention. Next generation sequencing technologies may be performed according to the manufacturer's instructions (as e.g. provided by Roche, Illumina, Applied Biosystems, PacBio, Oxford Nanopore or MGI).

In a preferred embodiment, the sample is treated by converting DNA methylation using enzymatic reactions, performing whole genome library preparation and measuring the methylation profile by sequencing (EM-Seq).

In a particularly preferred embodiment, the sample is treated by converting DNA methylation using enzymatic reactions, performing whole genome library preparation, hybridizing the whole-genome-converted library preparation to capture probes (preferably capture probes capable of capturing DNA regions in proximity to gene promoters); and measuring the methylation profile by sequencing (EM-Seq).

Method for Selecting a Dietary, Pharmacological, or Lifestyle Regime for a Dog

In a further aspect, the present invention provides a method for selecting a dietary, pharmacological, or lifestyle regime for a subject.

The dietary, pharmacological, or lifestyle regime may be applied to the dog for any suitable period of time. By way of example, the dietary, pharmacological, or lifestyle regime may be applied for at least 2, at least 4, at least 8, at least 16, at least 32, or at least 64 weeks. The dietary, pharmacological, or lifestyle regime may be applied for at least 3, at least 6, at least 12, at least 24, at least 36, at least 48 or at least 60 months. Suitably, the dietary, pharmacological, or lifestyle regime may be applied for at least 1, at least 2, at least 3, at least 4, at least 5, at least 6, at least 7, at least 8, at least 9 or at least 10 years. Suitably, the dietary, pharmacological, or lifestyle regime may be applied for the lifetime of the dog.

Suitably, the modification is a dietary intervention as described herein. By the term “dietary intervention” it is meant an external factor applied to a subject which causes a change in the subject's diet. More preferably the dietary intervention includes the administration of at least dietary product or dietary regimen or a nutritional supplement.

The dietary regime may be a meal, a regime of meals, a supplement or a regime of supplements, or combinations of a meal and a supplement, or combinations of a meal and multiple supplements.

The dietary intervention or dietary product described herein may be any suitable dietary regime, for example, a calorie-restricted diet, a senior diet, a low protein diet, a phosphorous diet, low protein diet, potassium supplement diet, polyunsaturated fatty acids (PUFA) supplement diet, anti-oxidant supplement diet, a vitamin B supplement diet, liquid diet, selenium supplement diet, omega 3-6 ratio diet, or diets supplemented with carnitine, branched chain amino acids or derivatives, nucleotides, nicotinamide precursors such as nicotinamide mononucleotide (MNM) or nicotinamide riboside (NR) or any combination of the above.

Suitably, the dietary intervention or dietary product may be a calorie-restricted diet, a high-calorie diet, a senior diet, or a low protein diet. Suitably, the dietary intervention or dietary product may be a calorie-restricted diet. Suitably, the dietary intervention or dietary product may be a low protein diet.

A dietary intervention may be determined based on the baseline maintenance energy requirement (MER) of the dog or dog breed. Suitably, the MER may be the amount of food that stabilizes the dog's body weight (less than 5% change over three weeks).

By way of example, it is generally understood that some dog breeds benefit from a high energy/high protein diet; however, other breeds may have a lower energy requirement and therefore diets can be appropriately modified.

Suitably, a calorie-restricted diet may comprise about 50%, about 55%, about 60%, about 65%, about 75%, about 80%, about 85%, or about 90% of the dog's MER. Suitably, a calorie-restricted diet may comprise about 60% or about 75% of the dog's MER.

Suitably, a low-protein diet may comprise less than 20% protein (% dry matter). For example, a low-protein diet may comprise less than 19% (% dry matter).

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

A dog food composition having a ratio of energy from protein to energy from fat below 0.80 may be advantageous to athletic dogs. A food composition high in protein and high in fat is particularly well adapted for athletic dogs. Typically, a dog food composition for athletic dogs has from about 20-30% protein and from about 15-25% fat. Indeed, a food composition dense in energy from fat will provide an athletic dog with sufficient energy for the moderate to very intense activities (i.e., brisk walk to fast run) in which it spontaneously gets involved. Furthermore, the energy from protein to the energy from fat ratio is found to be advantageous in such a food composition for maintaining the lean body mass of athletic dogs.

Similarly, a particularly well adapted robust dog food composition may have the ratio of energy from protein to energy from fat in such a food composition greater than 0.80. More specifically, a protein content from about 20-30% and less than about 15% fat. Because of their low resting metabolic rate, such a food composition is ideally adapted to robust dogs. The composition will have the effect of limiting the fat intake of robust dogs and therefore their tendency to be overweight.

The modification in lifestyle may be any change as described herein, e.g. a change in exercise regime.

Similar to a dietary intervention, the determination of breed contribution of a breed that generally benefits from large amounts of exercise may allow a determination a switch the test dog to an appropriate exercise regime.

Ideal activity level and type may differ according to breed or breed categorisation. For example, a robust dog will be spontaneously engaged in mild (e.g., slow walking), moderate (e.g., brisk walking) or occasionally intense (e.g., running) activity types. An athletic dog, in comparison, will mainly be voluntary involved in moderate, intense or very intense (e.g., fast running) activities. Within these different levels of activity, dogs can be further classified as robust or athletic.

The pharmacological regime may refer to administration of a therapeutic modality or regimen. The modality may be a modality useful in treating and/or preventing—for example—arthritis, dental diseases, endocrine disorders, heart disease, diabetes, liver disease, kidney disease, prostate disorders, cancer and behavioural or cognitive disorders. Suitably, prophylactic therapies may be administered to a dog identified as being at risk of such disorders due to breed contribution of a breed which is associated with that disease. In other embodiments, dogs determined to be at risk of certain conditions due to breed contribution may be monitored more regularly so that diagnosis and treatment can begin as early as possible.

The present invention may thus advantageously enable the identification of dogs that are expected to respond particularly well to a given intervention (e.g. dietary, pharmacological or lifestyle regime). The intervention can thus be applied in a more targeted manner to dogs that are expected to respond due to their breed profile.

Use of a Dietary Intervention

In one aspect, the present invention provides a dietary or pharmacological intervention for use in treating and/or preventing a disease in a dog, wherein the dietary intervention is administered to a dog with a breed profile determined by the present method.

As described herein, the dietary intervention may be a dietary product or dietary regimen or a nutritional supplement.

Computer Program Product

The present methods may be performed using a computer. Accordingly, the present methods may be performed in silico.

Suitably, the computer may prepare and share a report detailing the outcome of the present methods.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine the contribution of a dog breed to a test dog genome as described herein.

In another aspect, the present invention provides a computer program product comprising computer implementable instructions for causing a device to determine the contribution of a dog breed to a test dog genome; and optionally select a suitable dietary, pharmacological, or lifestyle regime for the dog based on the contribution of a dog breed to the test dog genome determined using the DNA methylation profile. The computer program product may also be given additional parameters or characteristics for the dog. As described herein, the additional parameters or characteristics may include age and sex of the dog.

In one embodiment, the user inputs into the device levels of one or more of DNA methylation markers as defined herein, optionally along with age and sex. The device then processes this information and provides a determination of a breed profile for the dog. Alternatively, the device then processes this information and provides a determination of a suitable dietary, pharmacological, or lifestyle regime for the dog based on the breed profile.

The device may generally be a server on a network. However, any device may be used as long as it can process biomarker data and/or additional parameters or characteristic data using a processor, a central processing unit (CPU) or the like. The device may, for example, be a smartphone, a tablet terminal or a personal computer and output information indicating the determined breed profile for the dog or a determination of a suitable lifestyle or dietary regime for the dog based on the breed profile.

Those skilled in the art will understand that they can freely combine all features of the present invention described herein, without departing from the scope of the invention as disclosed.

### EXAMPLES

The invention will now be further described by way of examples, which are meant to serve to assist the skilled person in carrying out the invention and are not intended in any way to limit the scope of the invention.

### Example 1—Illustrative Method for Distinguishing Dog Breeds Using DNA Methylation

Identification of DNA Methylation Sites

Whole blood samples from a canine cohort were analysed by performing DNA extraction, converting DNA methylation by using enzymatic reactions, performing whole genome library preparation, hybridizing the whole-genome-converted library preparation to capture probes directed against gene promoters and measuring the methylation profile by sequencing (EM-Seq).

The capture probes were directed against approximately 40,000 targets (promotor regions—approximately 1kb upstream and 0.5 downstream of transcription start sites). These target regions comprise potential methylation sites of interest (individual cytosine residues that may be methylated).

The following bioinformatics steps were performed after sequencing and before further analysis:
Quality check of fastq using fastQC—https-/www.bioinformatics.babraham.ac.uk/projects/fastqc/
Adapter trimming using trimGalore—https://www.bioinfornatics.babraham.ac.uk/projects/trim_galore/
Align to dog genome using bwamet or Bismark—(https://github.com/brentp/bwa-meth or https://www.bioinformatics.babraham.ac.uk/projects/bismark/)
Mark Duplicates using Picard—https://gatk.broadinstitute.org/hc/en-us/articles/360037052812-MarkDuplicates-Picard-
Call methylation using Methyldackel—https://github.com/dpryan79/MethylDackel

The methylation sites may be further filtered by (i) removing sites that are (un)methylated in all samples and/or (ii) removing sites that do not have at least 5 counts in at least 90% of the samples.

Distinguishing Different Breeds

A subset of samples composed of 66 samples from 2 breeds (Beagle and Labrador retriever) was analysed. 5000 sites were randomly selected, and the methylation beta value (percentage of methylation for each site) was determined for each dog.

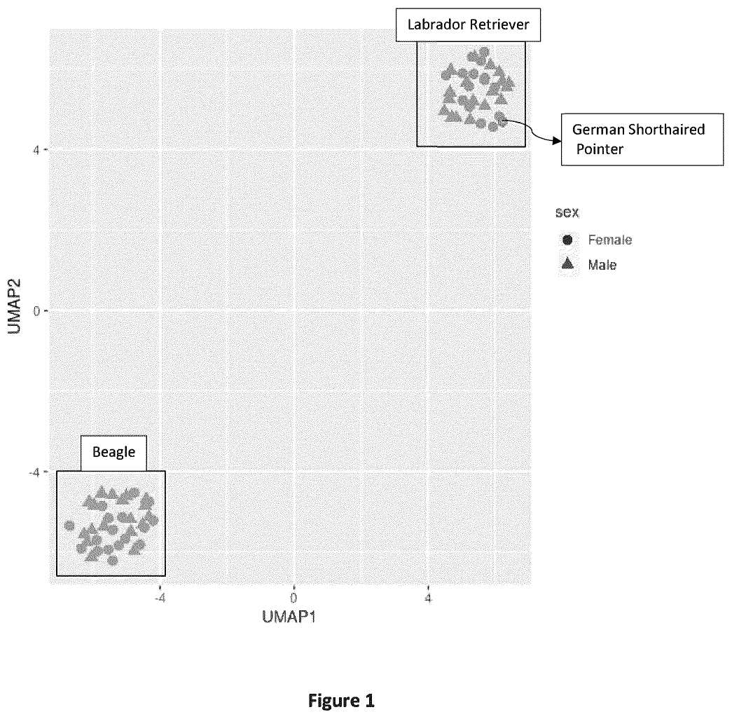

A binomial GLMnet (elastic net penalized binomial regression) was fitted to ⅔ of the data (training set) and parameters were estimated in order to maximize AUC (area under the receiver operating characteristic curve). The LASSO component of the model acts as variable selection and selecting alpha=1(LASSO regression) and lambda=0, we were able to identify 19 sites that can correctly classify the two breeds (see FIG. 1) with an accuracy of 1 on the testing set.

The 19 sites and their coefficients are presented below (chromosome and position of the site on the chromosome).

TABLE 5

| | |
|---|---|
| (Intercept) | −5.330946344 |
| chr30_29355719 | 1.934616743 |
| chr28_25696070 | 2.027095492 |
| chr17_6967618 | 0.267649728 |
| chr5_19735916 | 1.017842364 |
| chr6_19293329 | −0.540643196 |
| chr30_38142835 | −3.817854714 |
| chr38_798068 | −0.226793621 |
| chr31_14039889 | 0.003583211 |
| chr22_60248229 | 1.872789892 |
| chr18_25264409 | 1.117409783 |
| chr26_29530506 | 1.039490356 |
| chr23_47113160 | 0.414103225 |
| chr24_22274778 | 1.38832098 |
| chr20_57866776 | 0.780119944 |
| chr5_15661321 | −0.672923623 |
| chr11_72644485 | 10.41748845 |
| chr6_44119858 | 1.814787663 |
| chr13_38053859 | 0.672124037 |

### Example 2—Approaches for Filtering Sites to Generate a Restricted DNA Methylation Profile for Training Breed Identification

Following determination of the methylation status of methylation sites in each sample (as per Example 1—“Identification of DNA methylation sites”), the initial methylation profile is filtered/processed in order to generate a restricted methylation profile comprising fewer discrete methylation sites. The goal of this filtering is to provide a restricted methylation profile comprising e.g. between 50000 and 500000 methylation sites that can be used to train the method.

Methods for Filtering the Initial Methylation Profile Include:
1) Removing sites that are (un)methylated in all samples (e.g. % methylation=0 across all samples or % methylation=1 across all samples)
2) Removing sites that do not have at least 5 counts in at least 90% of the samples
3) Removing chromosome X and/or Y sites

Further potential filtering steps to reduce the number of discrete methylation sites include:
1) Defining methylation sites as DNA regions (CpG islands, sites that are less than e.g. 1000 bp apart are considered as the same)
2) Restrict targets to e.g. inflammatory sites, for example genes associated with inflammation from GO/KEGG pathway and select the sites on these genes
3) Correlation analysis and retention of a representant(s) per cluster (e.g. Weighted correlation network analysis (WGCNA) (Langfelder & Horvath; BMC Bioinformatics; 9(559); 2008).
4) Differential methylation analysis to pre-select DNA methylation sites (e.g. using logistic regression) or identify sites that vary more between different breeds (e.g. using variance analysis). Normalization (quantile) is performed before the filtering step if differential methylation analysis is performed (using function preprocessQuantile in R).
5) Reduce correlations and therefore dimension of the features space using e.g. EBmodule (Zollinger, A., Davison, A. C., & Goldstein, (2018).; Biostatistics, 19(2), 153-168. https://doi.org/I0.1093/biostatistics/kxx032),
6) Mutual information processing to select top sites Identification of breed contribution based on DNA methylation A dataset including information on the breed, chronological age, and sex of a cohort of dogs is split into training and testing sets (e.g. ⅔ data for training and ⅓ for testing, ensuring a good split with regards to metadata; for example similar proportion of each breed/each sex in the training and in the testing set).

A prediction model is built using a multinomial GLMnet (elastic net penalized binomial regression) to model breed as a function of the methylation profile. Parameters of the model as well as the penalty parameter are estimated using the glmnet package in R. Parameters of the model are tuned by using CV (10fold) and variables selected based on their importance in model contribution.

The penalization parameter is selected to provide a reasonable number of DNA methylation sites (e.g. max 100) with a good model fit for breed.

The model for breed identification based on DNA methylation sites is then assessed on the testing set. Models are assessed based on kappa, F1, precision and recall.

### Example 3—Further Dog Breed Classification Using DNA Methylation

Using a pet cohort composed of 829 dogs and 20 breeds, we developed a breed classifier exclusively using DNA methylation. The methylation beta-values at sites near promoters were obtained by sequencing blood samples. The lowly covered (<15) and missing values were first imputed using the Boostme algorithm (Zou, L. S., Erdos, M. R., Taylor, D. et a/.; BMC Genomics 19, 390 (2018). 1, a tree-based machine learning algorithm.

The X chromosome was removed. The sites were selected to reduce correlations and therefore dimension of the features space using EBmodule (Zollinger, A., Davison, A. C., & Goldstein, (2018).; Biostatistics, 19(2), 153-168. https://doi.org/10.1093/biostatistics/kxx0321. We separated the dataset into blocks of about 5000 sites by grouping the sites per target (1500 bp around the TSS) and per chromosome. Then we calculated a correlation matrix on which we apply EB modules as described in Zollinger et al. above. Each module is then represented by the medoid site. Some sites do not belong to any modules and are called scattered sites. They are defined as described in Zollinger et al. The number of sites was reduced to 471K from 1.4mio.

The training cohort was split into two subsets: training and testing sets. The training set comprised of dogs that potentially shared a familial relationship, while the testing set contained dogs unrelated to those in the training set. To ensure an adequate representation of each breed in the training set, we excluded breeds with less than four samples. In total 16 breeds of the initial 20 breeds were included. Furthermore, for breeds with fewer than ten dogs, we manually ensured that only one dog was included in the test set, while the remaining dogs were allocated to the training set.

To identify the relevant methylation sites for breed prediction, we used Mutual Information (MI) to estimate the predictive power of the sites. The sites were ranked according to the computed values, and the top N sites were selected. N was determined empirically, by fitting multiple classifiers with a range of N. As it performed the best according to the average F1 across breeds, we selected the top 200 sites as input for the classifier (see Table 1). Additionally, the best model, Support vector machine (SVM) with a linear kernel was selected using the F1 score across breed as the performance metric.

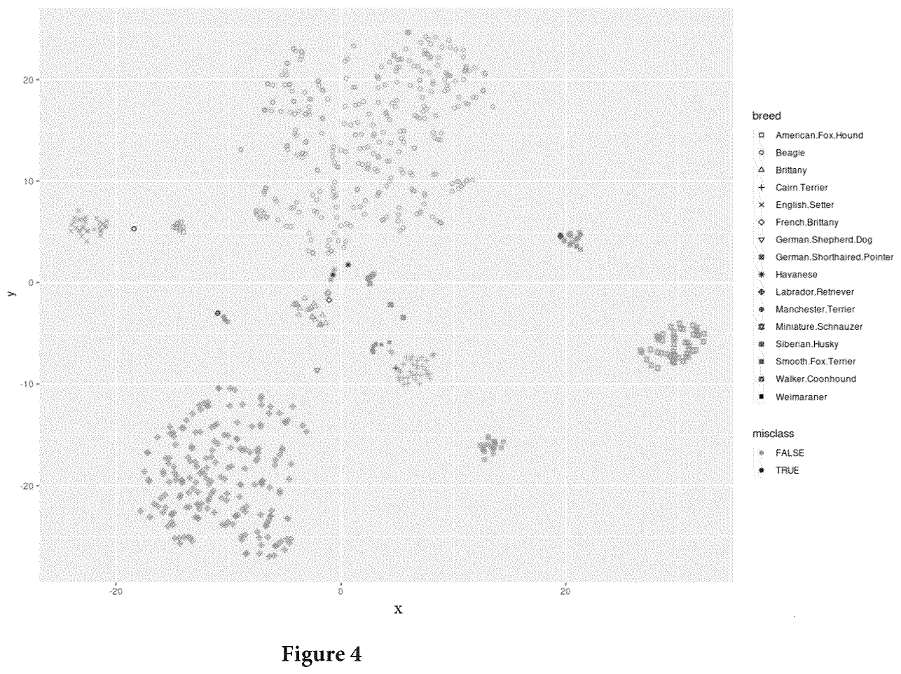

To mitigate the severe imbalance of the breeds distribution, different weights were given to the misclassifications of the different breeds. The weight was defined as the inverse of the breed fraction in the training set. The model achieved an average F1 of 0.89. Note that the cost hyperparameters of SVM was tuned to 0.1 using k-fold cross validation (k=10) (see Table 2 and FIG. 4). Moreover, to get probabilities from the SVM output for each breed Platt scaling was employed. Finally, to assess the generalizability of the model, we used a validation cohort, composed only of Labradors Retrievers. All Labradors were correctly classified.

Further classification models were also generated using only the top 5, top 10, top 20, top 25, top 50, top 100 and top 150 sites from the complete list of sites shown in Table 1; and each was shown to be predictive of breed classification (see Table 4). These classifiers were generated by selecting the top-n sites based on mutual information going from the most informative (top of the list) to the least informative. Average F1 score for the classifier using the top 5, top 10, top 20, top 25, top 50, top 100 and top 150 sites are shown in Table 4.

Description of SVM algorithm outputs:

To generalize to multi-class classification, SVM fit k(k-1)/2 binary classifiers and determines the class using a voting scheme. Meaning that for the 16 breeds, 120 binary classifiers were fitted. Moreover, a total of 340 support vectors (SV) were used to distinguish between all classes. For each of the SV, a coefficient is associated to each site (340×200). Then, for each of the class except one, a coefficient for the SVs is given (340×15) and the decision thresholds for each of the trained binary classification problem are returned (120).

Description of Provided Coefficients Using MLR

Multinomial logistic regression (MLR) and SVM with linear kernel achieved very similar performance. Therefore, coefficients of the multinomial logistic regression were provided. MLR takes the same 200 methylation sites as input. Similarly, differential weights were used to mitigate the class imbalanced. The weight for each data point is defined as the inverse of the fraction of the breed in the training dataset. The hyperparameters of MLR were tuned using cross validation on the training set: decay=0.1. To provide the coefficients of the model concisely, we fitted MLR with the same parameters, but only with the top 10 sites. The coefficients for each breed are given for the 10 methylation sites, in addition to the intercepts (see Table 3).

To calculate the probability of belonging to a breed class, one should calculate

$$p = \frac{{e}^{{β}_{0} + \sum {β}_{i} {x}_{i}}}{1 + {e}^{{β}_{0} + \sum {β}_{i} {x}_{i}}} ,$$

β₀ is the intercept, βᵢ, are the coefficients and xᵢ is the methylation value of each site i.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

TABLE 1

| | | Sequence | SEQ |
|---|---|---|---|
| Site | | (25 bases upstream and downstream | ID |
| Number | Site | of the 5mC site) | NO: |
| 1 | chr9:31701324- | CATCTACGCAAATGGTCTTCCCGTTACGTGGATTTTCATTGGCT | 1 |
| | 31701326 | CATCACCCT | |
| 2 | chr24:22575931- | AGCGCCTCTGCACCTGTTTCCCTGACCGGTATGCCTGACTCCC | 2 |
| | 22575933 | ATGGTGGGCT | |
| 3 | chr9:6627330- | TGAGGGCACCCTCGCTCGAACTCAGCCGGGGGTGTCCCTGGA | 3 |
| | 6627332 | GGGCCAGGCTC | |
| 4 | chr9:5238870- | AACACTTGCACATCCACGAGCAGGGGGGCGCTGCCATTGTTT | 4 |
| | 5238872 | GGAATCTGGAT | |
| 5 | chr11:65861798- | GAGTGCAGCCAAATAAGCAATTGGAGCGGGAAGGACTGCTCC | 5 |
| | 65861800 | CTGAGCTACGT | |
| 6 | chr3:74353518- | CAGAAGTTAGTAAGAACAGGTAAGGCCGTCTCCACGTCATCG | 6 |
| | 74353520 | TTTCTGTACAT | |
| 7 | chr5:63708131- | CACCACAGCAGCCTGAGCAGCCAAGGCGCCTGCCGCCACCAG | 7 |
| | 63708133 | CAGCATGACGG | |
| 8 | chr3:63619987- | CTGGCGATGGGCCTCCCCTGGCAGCCCGCTGGCCTTCACTATG | 8 |
| | 63619989 | CACAGGACCA | |
| 9 | chr5:15661221- | CCAAGACCACACAGTAAATTAGCGTCCGATTCCGAACAAATCC | 9 |
| | 15661223 | AGGTCTTCGG | |
| 10 | chr29:39142945- | AGGAACGACTCAGAAAGCACAGCCCACGGTGCACCCCGCGGC | 10 |
| | 39142947 | GCCCGCCCTCA | |
| 11 | chr1:116532571- | CCACCCTTTCAGAGGGACCCTCCTAGCGCCCTGTAAAGTGCTC | 11 |
| | 116532573 | TCCTCCCTCA | |
| 12 | chr3:63619179- | CAGGTCCACAGTGTGAGCACGGTGACCGAGGACGTGCGGAC | 12 |
| | 63619181 | ACCTTGGTGAGC | |
| 13 | chr2:52200689- | TCGCCTGGCAAAGACCCTTGGCCTCCCGAGATTTCAGGGTCCC | 13 |
| | 52200691 | TGGGGGGGCC | |
| 14 | chr27:44169039- | GGGAGGGCCTCGCCCCCGCAGCCTCTCGTCCCGCGGTCTCCCC | 14 |
| | 44169041 | CTCCGCACTT | |
| 15 | chr36:23293794- | GTGCCACCGCCGCTGCTCCTGCCTCCCGGCCCGCTGCACGGTC | 15 |
| | 23293796 | CAGGTCTTCC | |
| 16 | chr9:51698589- | GCAGCGTTTCGTTGAAAAGAAAGGTCCGTGGGGGAAGTGGG | 16 |
| | 51698591 | GACCCCGGTTAG | |
| 17 | chr26:38424335- | TTTCCCCGCCAGGCCCCCGGCTAATGCGTCCCCCGGCATTCGC | 17 |
| | 38424337 | CAGCGCTCAT | |
| 18 | chr9:1127907- | GCTCTGACGTGGAAGACAGGTCAACACGAGCGTCATCCAGGT | 18 |
| | 1127909 | GTACAACTGTA | |
| 19 | chr11:34245204- | CCCAGAGGACGCCAGGCTGACGGGGTCGAGGCCAAGGCCGC | 19 |
| | 34245206 | TACCCTTGAGGC | |
| 20 | chr17:59673930- | AGTGCCGACAGCCACGGGGCTGGGGGCGAGGGACGTCTATC | 20 |
| | 59673932 | CAGGGGGTTCCT | |
| 21 | chr12:35285363- | AGTCACGCTGCCAACGAGGCTAGGCCCGACCGAGTCGCCCGG | 21 |
| | 35285365 | CCACGGCGCTG | |
| 22 | chr18:52582598- | ATGAGCGGGTCAAAGAGCGCGCACGTCGCTCTGGCTCCCCCG | 22 |
| | 52582600 | CAACACCGCCA | |
| 23 | chr12:5864764- | GGGACCGCTGTGGACGTTTCCGTGGGCGTCCGCCTGCAACGT | 23 |
| | 5864766 | CCCTCGGCCCT | |
| 24 | chr17:1884303- | AGCGCCATCAGTAAACCCCACAGCTGCGGTCGCACACAGCAG | 24 |
| | 1884305 | AGCAGGCCAGG | |
| 25 | chr5:15661321- | CCCTGGCCCCTCCATGCCTGGGACCACGCAACCACGTGAGGA | 25 |
| | 15661323 | AACGTTTTGTT | |
| 26 | chr6:10992657- | GTAATAGAAGGTGAAATTGATGTGGACGGTGCCACCCTTGGG | 26 |
| | 10992659 | GGCGGAGAGGT | |
| 27 | chr21:40484402- | GGGCCTCCAAGCCCTGTTTCCATGTCCGGTACCTGCTCAAGGC | 27 |
| | 40484404 | ACAGGCGACC | |
| 28 | chr13:58147303- | CAAGGTCACGGACAGCCTGCGAGACTCGTCAAGGTCACGGAC | 28 |
| | 58147305 | AGCCTGCGTGA | |
| 29 | chr9:42468292- | CACAGGTGCTTAGTACAGGGCCCTTGCGCCCCACCTTGCTGCT | 29 |
| | 42468294 | GCGAACAAAA | |
| 30 | chr15:18330494- | GGCCAGCTGATGGGGTCCCCCTGGGACGGAGATTCTCTTGCC | 30 |
| | 18330496 | AACAAGTCAAG | |
| 31 | chr37:30911104- | GAAGTCTGATGATGGGAGACGGGCAGCGACTGCAGCGAGAC | 31 |
| | 30911106 | ACAGGGCATGTC | |
| 32 | chr11:34245198- | CTGCAGCCCAGAGGACGCCAGGCTGACGGGGTCGAGGCCAA | 32 |
| | 34245200 | GGCCGCTACCCT | |
| 33 | chr8:72319433- | CGGGCGCGCCCAGGCCGGGGCCCCGGCGGCCGACCATGGTG | 33 |
| | 72319435 | CTGCCGCCCCCG | |
| 34 | chr37:25536190- | TGCCCCACGCCCCACTGGGCCCCCTCCGGGCCCAGAAATGCG | 34 |
| | 25536192 | GCCTCCCCTCC | |
| 35 | chr28:2463302- | TCACTCAGTTTCCTATTCATCGTTCCCGCTCTCCACCCTTCCTTT | 35 |
| | 2463304 | CTGCCTCT | |
| 36 | chr29:36298422- | AGATCCTCCCGCCTCTCCGCAGGGCCCGCAGCACCGCTCCCGT | 36 |
| | 36298424 | TGGGGGTGCA | |
| 37 | chr10:19780509- | CGATGGGCCCAGAGGCTCCCTCCCTACGCTCCCACAACGCTCT | 37 |
| | 19780511 | GTAAACACCT | |
| 38 | chr7:220852- | TCACACACCAGCCCTCCGGGGAAGGCCGCGGAGCCGTCGCGC | 38 |
| | 220854 | CACCAGCGGTC | |
| 39 | chr17:61459518- | AGCTGGCAAGTAGCATGGGGAGCACCCGAATCTAGTCACCTA | 39 |
| | 61459520 | ACTTCAAATCC | |
| 40 | chr10:19780397- | CACACCCCACTCGGCACCACCACCCGCGGGTTCTCCCGGCAGT | 40 |
| | 19780399 | GCCTGCATTG | |
| 41 | chr7:767703- | TAGGAGAGAGTGGATGCCTTATCTTTCGTGATTTCCGCCAAGA | 41 |
| | 767705 | CAATTATGGG | |
| 42 | chr11:53827468- | AAGGGATGTGACTTGCCTACAGAGCACGATATTGGCCAGGTC | 42 |
| | 53827470 | AGGTCTCCCTC | |
| 43 | chr10:10975036- | GGCTGGAAGAGTGTCCTCCCCGCGAGCGGGGCTCCTGGCGG | 43 |
| | 10975038 | GGGGCGGGGGCT | |
| 44 | chr33:8298639- | CCGAGGTGTCAGGTCGTAGCTGGGGACGGGAGCGCGGCTCG | 44 |
| | 8298641 | GCACCTGGTGGG | |
| 45 | chr25:45106763- | TGAAAACCCAGCCTGCATTCCCCCTGCGCCATGTCCCTGTCCCC | 45 |
| | 45106765 | AGCCCTCCA | |
| 46 | chr35:10660004- | AGAAAGGCACGTGACTGTGCATGCGCCGACGGGAGGCGCGT | 46 |
| | 10660006 | GGGCAGAGAAAG | |
| 47 | chr38:22224552- | GGGGACTCAGGTGGGCACTCGGCGGGCGCTCAGGCGGGCAC | 47 |
| | 22224554 | TCGGTGGGCACT | |
| 48 | chr6:40350960- | TCTAAGACCCCTGTCAGCGCTGGGCCCGTGCGCAGGGAGGCG | 48 |
| | 40350962 | CGGAGCAGCAG | |
| 49 | chr28:40153258- | CTCCTGGGCCTCATGTTCAGGGGACACGGCACAGGGGTCTGG | 49 |
| | 40153260 | GTCCGTGGGCC | |
| 50 | chr2:70381095- | CCCCACCAGCTTCACCATCCATAACCCGTCTCAGGGCCATCCA | 50 |
| | 70381097 | CCCTGTCCTT | |
| 51 | chr26:19386077- | GCGGGGCTCCCCTCCTCCCCCGATCCCGGCCTCCAGTTATCTG | 51 |
| | 19386079 | GGTGAGGCAG | |
| 52 | chr24:21946951- | CATGAACCACGGTGAGTTGGTGGGCCCGGGTGTGGGGTTGG | 52 |
| | 21946953 | GGGCAGGTAGCA | |
| 53 | chr36:23294492- | AGGCCACCCTGTACCTAGAGGGGCGCCGGGGGGGGCTCGCG | 53 |
| | 23294494 | GGGTGCAGGGCA | |
| 54 | chr20:22370926- | AGGTGCTGCGGACGTGGTGCGGGCTGCGCGACGTGTACCAG | 54 |
| | 22370928 | GTGAGCCGGGCG | |
| 55 | chr26:13687899- | TGAAGCGGACACACCACGTGCCAGAGCGGCCCATCCCAGTGG | 55 |
| | 13687901 | CACAGCCCAGG | |
| 56 | chr9:1506547- | GCTCACTGGCACTTGGCGCCTCCACGCGGGGCAGGCTGACCC | 56 |
| | 1506549 | TGGGCAGCGCC | |
| 57 | chr17:56449224- | CCCAGGACTGTGAAGGCCTCATCGTCCGTTCGGCCACCAAGG | 57 |
| | 56449226 | TGACTGCTGAC | |
| 58 | chr2:75347954- | CGCATGAAAACCGACACACCTGGCCACGCGCACTTAAGTTTCT | 58 |
| | 75347956 | CAGCTTCTCT | |
| 59 | chr4:24311681- | CACCCCACTGGACAATGAGGTCTGCTCGGCTAAAAATATCCCT | 59 |
| | 24311683 | GAGCCACTAA | |
| 60 | chr31:38789728- | TCCTCACCGCAGGCTCCTACCTAACTCGATCCCGTCCTCACCGC | 60 |
| | 38789730 | AGGCTCCTA | |
| 61 | chr15:14161230- | ACACGGTGAACGCCTGGTCAGGACTCCGGGGACCCAGCCCTC | 61 |
| | 14161232 | TTCATTAGGGG | |
| 62 | chr17:36575147- | TCATCCCCATTTACAGATGAGCACACCGATTCTCCCAGCAGCT | 62 |
| | 36575149 | GTCACTTGCT | |
| 63 | chr13:37742526- | CAAGAGGCCCGCCCTGCGCTGGGGCGCGGCCGGGAGCTCAG | 63 |
| | 37742528 | ACCTGGCTCACC | |
| 64 | chr23:45188467- | CTCGACGCTGCTGTCCCCGAAGGCCGCGGCCCCCCAGAGGTA | 64 |
| | 45188469 | CGCATCGCTGA | |
| 65 | chr6:16132830- | GGCAGCTAGGTGCCCACCGCCACCTCCGCTGCTGCCCATGCA | 65 |
| | 16132832 | GGTCCCTGGGC | |
| 66 | chr28:40110981- | ATTTTTACCAGAGATATTCCCATTAACGTTAATTTGTAGACCAA | 66 |
| | 40110983 | GTCTGTCTA | |
| 67 | chr9:51877632- | TGGTGTCTGCACTTACTGTGATCTCTCGATTTCATGCTTCATCA | 67 |
| | 51877634 | AATATTCAG | |
| 68 | chr38:22153093- | TAGCTGGGAGACTCTCAGCGCAAGTCCGAAGGGCCGGCTTCT | 68 |
| | 22153095 | TGTGCCCCTCC | |
| 69 | chr3:40933518- | AAGAGTCCCCTGGGGCCTGGCGACTCCGAGTGAGTAGGACCT | 69 |
| | 40933520 | GTGGTTGTCAC | |
| 70 | chr5:62850824- | TGGAGAAGCTCAGCTCAGCCCAGCCTCGGCCTCCTTGCTGGTC | 70 |
| | 62850826 | TGACTGGTAA | |
| 71 | chr3:57431609- | AAGCCCTCCGATCCGCCCGATCGGCCCGGGCTCCCCGTCCTCG | 71 |
| | 57431611 | CACCTCCGCA | |
| 72 | chr20:44688803- | GAGGTGCTTCTGAACCCCAGTCTCTGCGCTTCCTGCAGCTAGC | 72 |
| | 44688805 | TCCTCGCGTT | |
| 73 | chr3:94100812- | GGGTGGTGTGCAGCTGTGTCACCTCACGTCCTGGCCACCTGCC | 73 |
| | 94100814 | CAGAGCTGGC | |
| 74 | chr11:49988106- | CTTGGGAGGAGGCCCAGCACCCATCCCGCATCTTGGTCCTTTG | 74 |
| | 49988108 | GGATTCAACC | |
| 75 | chr18:37672016- | TTGGCAATCATGGCCTACGATCGCTACGTGGCCATCTGCCACC | 75 |
| | 37672018 | CGCTGCACTA | |
| 76 | chr5:58166170- | CCCGGAACCTGCAGCCGGGAAAGGCCCGGGTGTCCAGGAAA | 76 |
| | 58166172 | GCCCTGTGCCTC | |
| 77 | chr5:8606892- | TGGCCCAAGCTACCAGCCTCTCCCACCGAGACTACTGGTCTCC | 77 |
| | 8606894 | CACTGCTCTC | |
| 78 | chr18:50744464- | TGGTGCAGCTGCTGCTGCAGCGCTGGCGGGACCCGGAGTCAG | 78 |
| | 50744466 | GCCTGGACTCA | |
| 79 | chr21:5975549- | GCTCCGGGCCTCCAGGCCGCGGCCTCCGTGGGGCTGGTGCGC | 79 |
| | 5975551 | GCTGCAGCCGC | |
| 80 | chr28:34337846- | CCAAAGCTAGGGTAGCAGTCGCTAGACGCTGCCGAAACCCCA | 80 |
| | 34337848 | GGCCTGACGTT | |
| 81 | chr26:10521237- | AAGTCTCCACCCCGCAGTGGACTATCCGCCTAGTTTTGTGCCA | 81 |
| | 10521239 | GTCCCAAAGG | |
| 82 | chr28:40110757- | AGCAAAGGGTGTTATTTGGGGCATGCCGGGATCAGCACTGCT | 82 |
| | 40110759 | TTAACCCGAGG | |
| 83 | chr18:53611814- | AGGGGTGCAGGACGTGAGCAAGAAACCGCGCTCTCCTGCAA | 83 |
| | 53611816 | GGCTCTGTTTAA | |
| 84 | chr31:38789762- | TCCTCACCGCAGGCTCCTACCTAACTCGATCCTGTCCTCACTGC | 84 |
| | 38789764 | AGGCTCCAA | |
| 85 | chr18:37643689- | TTGGCAATCATGGCCTACGATCGCTACGTGGCCATCTGCCACC | 85 |
| | 37643691 | CGCTGCACTA | |
| 86 | chr13:58147037- | CGTCTGCTACAGGCCGGGCACCGCCACGGATTTAATAGGGTC | 86 |
| | 58147039 | GTGATTTTTAG | |
| 87 | chr1:97278257- | CGGACCCCTCGGCAGGGATGCGCATGCGTCCTGTCCACTGGC | 87 |
| | 97278259 | CGCACCGTGGA | |
| 88 | chr9:19008173- | ACTCACACCTGTCGAACACCTCAATCCGTGCTCCTTCCTCTGTG | 88 |
| | 19008175 | TCAAGCTGC | |
| 89 | chr6:60068413- | CTAGAAAAGAGCAGATOCTGACCTACCGTGGTCACACTAGAT | 89 |
| | 60068415 | GCTGGGCACAT | |
| 90 | chr20:57060644- | TGGTGGGGTCGCACTGCGGGCTGGCGCGGGTCCCTCCAGCCC | 90 |
| | 57060646 | ATCCCTCCCCG | |
| 91 | chr13:37816776- | AGGCAGGGACAGACAGGGGGTTCGCTCGGGGGCTCCTTCCTC | 91 |
| | 37816778 | TAGGGACAGAG | |
| 92 | chr17:56448390- | GGTCACAGACATAGGCTGCCAGTAGACGCGATCAATAGGTGC | 92 |
| | 56448392 | TGGTTGAATAT | |
| 93 | chr8:71674666- | AAGGCCGGCCGCGCCGCACTCTGGGGCGCCCCCCTGCACCCA | 93 |
| | 71674668 | GCAGACACCCA | |
| 94 | chr38:21444401- | GGGCCTGTGAGTGTGCATGGTCGGGTCGGGGCTGAGCGAAC | 94 |
| | 21444403 | TAAGCCTTGGGG | |
| 95 | chr10:19780028- | CACCACGGCCAACAGCCTGGGCTGCACGTCACTTGCCTGGCC | 95 |
| | 19780030 | GCCATGGCAGT | |
| 96 | chr1:113430519- | TCTCTGACATGTCTGTCTCCTCCATTCGTTCACACATTTCCTGA | 96 |
| | 113430521 | GCTCCCAGC | |
| 97 | chr1:116656331- | CTTCCTGGTCAGGCCCTGTCCCCTGTCGCTTCTCCAGAAAAGC | 97 |
| | 116656333 | AAGCACCAAA | |
| 98 | chr6:41381990- | TTAGGGCACTGCATGCGAGCCCGGGGGGGGGGGGGGCACGG | 98 |
| | 41381992 | CAGCCACCGGGG | |
| 99 | chr14:36261206- | TTTCCATGGGCTGTTTTTATTTTCTCCGCATTTTCTCCCTACTCT | 99 |
| | 36261208 | TCTTCTTC | |
| 100 | chr2:55675527- | CTGTCTCCAGGCTTTTCCTCACCTCTCGCCATATCCTGCTGATTC | 100 |
| | 55675529 | TGCTTCCT | |
| 101 | chr16:2123852- | CAGGGGGAGCGAGGAGGAGTGGCCAGCGAGACTCTCCAGAC | 101 |
| | 2123854 | CAACGACGTGCT | |
| 102 | chr1:22114976- | GTGCAATGAACCGGTTCAATCCACCGCGCTTGTTGTGGTTTGA | 102 |
| | 22114978 | AGGTGAGTGC | |
| 103 | chr9:5393488- | AGCACACTGCCCCAGGGCAGGTGCCGCGAGGATACCGGGCTT | 103 |
| | 5393490 | CCAGAAGAGCT | |
| 104 | chr13:37305361- | CCGGGGGCTCTGGGCCTCCTGAGCCCCGCGGGGTGTGTGCGT | 104 |
| | 37305363 | GCGTGTGCACG | |
| 105 | chr20:54910128- | TCAGCAGCTTCCACTTCCACCCCTGCCGGGAGCTGCTTGTGCA | 105 |
| | 54910130 | GTTTGTTGGG | |
| 106 | chr26:1578300- | AGCCGGCTCCTCTGCTCTGACTCAGCCGCAGCCCTGCTGCCTC | 106 |
| | 1578302 | TGGCCCAGCA | |
| 107 | chr11:34245187- | CTAGTGCCAGACTGCAGCCCAGAGGACGCCAGGCTGACGGG | 107 |
| | 34245189 | GTCGAGGCCAAG | |
| 108 | chr5:68353413- | TGGGCCTAGAGGCCACAACTGCTTCTCGAGGTCACTGAAGGC | 108 |
| | 68353415 | CCCCCACATCC | |
| 109 | chr2:62025395- | GACTTGGTAACCTGTCTCCCTTCACCCGCGCTGCCTCCTCAGAT | 109 |
| | 62025397 | CCACGAAAC | |
| 110 | chr5:9408111- | TGATTTATGGATGTTTTGGACTAACCCGCCCTACATAGCTTCG | 110 |
| | 9408113 | GAACTAGATC | |
| 111 | chr11:53827525- | GGGGCGTGGCTTCTTTCCTTCTACTGCGTCTTTTGTTATCTGCA | 111 |
| | 53827527 | ATCTTGGAC | |
| 112 | chr15:9451596- | GACTGCCCACTGTTAGGCGGCCTCTCCGGGCCTGGCGCTGAG | 112 |
| | 9451598 | GGGTGCTTGCC | |
| 113 | chr28:35778234- | GCCGCCTGCCGTCCCTCCGTCTCCGGCGCCTCTACCTGTGTTCT | 113 |
| | 35778236 | CGGGTCACC | |
| 114 | chr13:38137301- | CAGCCTTTTCAAATCCCAGAGATACGCGCGCATGAGCAGACCA | 114 |
| | 38137303 | CCCGGTTTGT | |
| 115 | chr13:38440940- | CGGCGCGACCCCCGCGCGACTCCTCACGGCTGCGGTGCTCTC | 115 |
| | 38440942 | GCCCTTCACAG | |
| 116 | chr2:56307502- | TGTTTCCGGAAGGCAGCCAGAGGGAGCGCTCAGGGCCTTTGC | 116 |
| | 56307504 | ACCTGCTCGAA | |
| 117 | chr3:86522254- | TCCGGCCCATCCTCTGGCTTGACTTCCGTCGTGGTGCTTAATTC | 117 |
| | 86522256 | TCCCCGCAA | |
| 118 | chr24:25406117- | AGTGGTTCTGTGACGGCGGGAGGCTTCGGGACGCTTTCCTGT | 118 |
| | 25406119 | GCTCCGTGAGT | |
| 119 | chr2:81022157- | CCAGAACTTGGGGTCAGTCCCTTGCCCGGAGGCCCCACCTCCG | 119 |
| | 81022159 | CCCCCCAAGG | |
| 120 | chr5:68349717- | AGTTCACAGAATATCGCCGACTTATCCGGCTAATTCCCAGACC | 120 |
| | 68349719 | CACACCTCAA | |
| 121 | chr38:22777777- | CTCAGCCACGCCGCCTCCTTCGGGGCCGGGGCCCTCTGGCTGC | 121 |
| | 22777779 | GCGGGCCCCG | |
| 122 | chr5:14580578- | TAGGCAAAGGGGGAAAGGGCCGTAATCGACCTGGCTCTTCTC | 122 |
| | 14580580 | CACTGGATGGG | |
| 123 | chr5:68352734- | AAGGGATTCCACCTGCTTACAGGCCCCGGAAGCTCCCCTGGA | 123 |
| | 68352736 | ACCCACATCAC | |
| 124 | chr30:37750648- | TCGAGGGAAAGTGAGCTGCTCCTGGCCGGCCCGCCCTCTCCCT | 124 |
| | 37750650 | GGCCCTGGCA | |
| 125 | chr27:43319920- | AGTTGTTTACCAAGGCCTGGTTCTCACGTGTGTTCTGGCCTTC | 125 |
| | 43319922 | GCCCTCGGCC | |
| 126 | chr8:3698136- | GCTCTAGGGAGAGAAGAGCAAGCTACCGACCCAGGAATCCTA | 126 |
| | 3698138 | CAGATCAGACC | |
| 127 | chr31:38789801- | ACTGCAGGCTCCAACTGACTCGATCCCGTCCTCACCGCAGGCT | 127 |
| | 38789803 | CCTACCTAAC | |
| 128 | chr27:43716868- | AGGGGGGCCAGGAGGTTCTTGTTGACCGTGACCTGATGGATG | 128 |
| | 43716870 | CCCCCCGGAGG | |
| 129 | chr38:21444411- | GTGTGCATGGTCGGGTCGGGGCTGAGCGAACTAAGCCTTGG | 129 |
| | 21444413 | GGAACTTTGCGC | |
| 130 | chr5:9441510- | CCCTCGCCTCCCCCCACTTCTCATCCCGAGGAACAGTCTTGGG | 130 |
| | 9441512 | ATGGAAAGAA | |
| 131 | chr38:22224692- | ACTCGGCAGGTGCTCAGGTGGGCACTCGGCGGGCGCTCAGG | 131 |
| | 22224694 | CGGGCACTCGGT | |
| 132 | chr12:522114- | AAAGAAGCAGAATCAGCAGCAGCCGCCGCCTCCGCAACAGCC | 132 |
| | 522116 | CCAGCTGCCAG | |
| 133 | chr20:57685631- | GGTTCCTTCACCCTTACACGGGGGCCCGAGTGGCCGTGGCCC | 133 |
| | 57685633 | CCCTGCCAGAG | |
| 134 | chr18:50671536- | TTGGCCTCCTCACCTGGAGCTTGGGCCGCTCTGATACCCTCCT | 134 |
| | 50671538 | AGGCAAAATT | |
| 135 | chr25:26751246- | CACAGCCCGCAGAGCCTTTCACTTCTCGGGGGATGATGTCGCT | 135 |
| | 26751248 | CACTCCTAGA | |
| 136 | chr36:18662444- | CGAATTGCCCCGGCCCGTCTCAAGTTCGCACGCAGGCTGCTG | 136 |
| | 18662446 | GGTTCCGTGTC | |
| 137 | chr20:55800228- | ACCCTTGGGTTTTTGTCAACAGCAACCGGAGGTGACGTTCTTT | 137 |
| | 55800230 | TCTCACACTT | |
| 138 | chr9:19502614- | TGCAGCAGCGCCGTCTTGCCGCACTCCGCGTCCCCCACCACCA | 138 |
| | 19502616 | CGATCTTGCA | |
| 139 | chr17:20324242- | GTGAGTTCACTGTATGTCAGCCAGCACGTCAGAGCCAGCGTCT | 139 |
| | 20324244 | CTTCTCACCC | |
| 140 | chr20:55471151- | CTCCAAAACCCCAGGCAGGTGGGATCCGACAGTCAAGATCTG | 140 |
| | 55471153 | TCCCCTTCCAA | |
| 141 | chr28:25615324- | GCCACCCAAGGGTGTGCCGCAGCCAGCGCAGGAAGAACGAG | 141 |
| | 25615326 | AAGTTTCCTGCA | |
| 142 | chr31:38789743- | CCTACCTAACTCGATCCCGTCCTCACCGCAGGCTCCTACCTAAC | 142 |
| | 38789745 | TCGATCCTG | |
| 143 | chr7:37654841- | TTGCGACCGATGACCTGTGAATTGGCCGAAGGAGCTGTGACT | 143 |
| | 37654843 | CCTGGATTTGG | |
| 144 | chr4:28997945- | CTCTTTCTCCTTCTGCACAAGGGAGACGGCCAATAAACAACAG | 144 |
| | 28997947 | TGAGTTTGCC | |
| 145 | chr32:5525788- | ACCCTGCAGCACAGCGTCCCCTTCCCCGCGGCCGAGGAACGA | 145 |
| | 5525790 | GCGCCGGAACC | |
| 146 | chr9:60006132- | AATTCCACCCCAACCCCAGGAGCTCCCGGCCCCACGGAAGAG | 146 |
| | 60006134 | ACAGTACAGCG | |
| 147 | chr25:50466983- | CCTACCTCCTCGGACAGCATCGCCCCCGACCCCGCGAGCCCTG | 147 |
| | 50466985 | AGCGCTCCCC | |
| 148 | chr38:22224669- | ACTCGGCGGGGGCTCAGGTGGGCACTCGGCAGGTGCTCAGG | 148 |
| | 22224671 | TGGGCACTCGGC | |
| 149 | chr13:38440060- | GGAGGCCTCGGAAACAGCGGAGAACACGGTGACCTTTCAAG | 149 |
| | 38440062 | GAGATGCAGGGA | |
| 150 | chr10:19780454- | GCGGCCTTTGGAGTGACTCCCATCTCCGTGCCAGCCGCCGGAC | 150 |
| | 19780456 | TTGGGGGCCG | |
| 151 | chr24:33364927- | CCACAAGATCAACAAAGAATCTATCCCGTTCTTCCAGCCCATC | 151 |
| | 33364929 | GCCAAGGCCT | |
| 152 | chr37:26964674- | TACCACCTGTCATCACCCACGGGGCTCGGTGCCCAGGCCGGG | 152 |
| | 26964676 | GGCATCACTGA | |
| 153 | chr7:66488656- | TACTAGATGCAACCTAGGGTCGCAACCGGTTGTTAGAAACAA | 153 |
| | 66488658 | CACTAATACAT | |
| 154 | chr18:50259338- | ACACGCCCGGGTAGGGTACATGACGCCGGGGGCCAGGGGCG | 154 |
| | 50259340 | CGGAAAGCAGAG | |
| 155 | chr28:40800330- | GCAGCGGGGCCCAGCAGCGCCGAGGGGGGCCACCCCATCAG | 155 |
| | 40800332 | TGGGGCCACCGC | |
| 156 | chr15:14300072- | CTCTGCGCTGGGGCCCCGGGGGGCTCCGCTGCTCCCCCGCCCC | 156 |
| | 14300074 | AGGGGAGTCT | |
| 157 | chr2:75253761- | ACTGCGGTTGGCTGAGCAGATGCCTACGGTATTTTGGACACA | 157 |
| | 75253763 | AATTACAGAAA | |
| 158 | chr10:10975030- | CCCGAGGGCTGGAAGAGTGTCCTCCCCGCGAGCGGGGCTCCT | 158 |
| | 10975032 | GGCGGGGGGCG | |
| 159 | chr38:21444318- | TGTGCGTGCAGGCTGCGGCGAGTGTGCGTGCTCGGCTGGGAC | 159 |
| | 21444320 | CTGAGTGTACA | |
| 160 | chr11:52338302- | AAGGGCCAAGGCCGCTCAGAACAAGTCGCATTCCCTGAGGGC | 160 |
| | 52338304 | TTGATCATGCG | |
| 161 | chr5:42348549- | GGGGAGGCGCCGTCGGGGCCCCCAGCCGGCTGCCGGTGCAG | 161 |
| | 42348551 | CCTGCCCCACGG | |
| 162 | chr7:40016120- | TCCTAGTGGTGCACCCTCTGGCTCACCGCCTCCACGTTAGACC | 162 |
| | 40016122 | TCACCCCCAT | |
| 163 | chr1:101486848- | ATCATCTACCTCATCTGCAGTCACAGCGAGCTCCACAAGCCCA | 163 |
| | 101486850 | TGTACTTCTT | |
| 164 | chr8:71880556- | TTGGACGTCAGGGGGGGGATCTGCGCCGGGCCTTTCGCGTCC | 164 |
| | 71880558 | AGGGGCTGGGA | |
| 165 | chr2:78822683- | GATTTTCATGCCACGTTCTCTAACTACGTTTGAAGTTAGGCAG | 165 |
| | 78822685 | GCAGGGCTTA | |
| 166 | chr38:21444396- | GGTCAGGGCCTGTGAGTGTGCATGGTCGGGTCGGGGCTGAG | 166 |
| | 21444398 | CGAACTAAGCCT | |
| 167 | chr6:20796027- | TCACGGTCTCAGCTATGACGAGGGCGCGAAACCCCTCGATGT | 167 |
| | 20796029 | CCTCCCACTCT | |
| 168 | chr30:38142713- | GTGGGGGGTGTATGTGGTGGGAACGGCGCGGTGCTGGGGCC | 168 |
| | 38142715 | CGGAGGCCTCCC | |
| 169 | chr2:78822438- | AACTCCAGTCACATGGGTCTGCCTTCCGTCCCCTGCACACTGTC | 169 |
| | 78822440 | CGGTCTCCC | |
| 170 | chr20:7419572- | ACACCGCCTGCAAGTCCTGGCACCGCCGCCCCCTTCCATTTTCA | 170 |
| | 7419574 | CAAAGGCTC | |
| 171 | chr31:38789795- | GTCCTCACTGCAGGCTCCAACTGACTCGATCCCGTCCTCACCG | 171 |
| | 38789797 | CAGGCTCCTA | |
| 172 | chr21:10119352- | AAGCTAATCCCACACACCAGTGTGCCCGCAGTAGCTGCAGCT | 172 |
| | 10119354 | GGGCCTCTCAG | |
| 173 | chr1:24915725- | CTCTGCACCCCAGCTCCTCCGCACCCCGGCTCGCGGTCAGGAG | 173 |
| | 24915727 | ATGCCCAGGA | |
| 174 | chr24:22156413- | AGCCCCTTCCAAAACCACACTGTGCCCGGTCCAGCTCCTCTGC | 174 |
| | 22156415 | CATGTGCCCC | |
| 175 | chr6:41653487- | GTCTCCAGGCCCCCCAGGGGCTCCAGCGCTCGCAGGAGGACG | 175 |
| | 41653489 | CACGCCCCATC | |
| 176 | chr31:38021442- | GGCCCGCGGCCTTCGGGCGGGCAAGCCGCGGGACTGACAGG | 176 |
| | 38021444 | CCAGCACAGTGG | |
| 177 | chr2:75295704- | CAGGGGACTCCTTGCTTGGAGTTTAACGGTCGAGACAGCACA | 177 |
| | 75295706 | GGAATGAGCAC | |
| 178 | chr32:41016395- | GTGCGGCGACCGTGTGACCGTGTCCCCGCGAGCAGGTGCGCT | 178 |
| | 41016397 | CGTCCAGGGCG | |
| 179 | chr37:25536217- | GGGCCCAGAAATGCGGCCTCCCCTCCCGCAGCCACAGCCCCC | 179 |
| | 25536219 | GTAGCCAGCCC | |
| 180 | chr8:44230723- | CAGCCAGTGAGGCTGCTGATCAAGGTCGGCCAGGGAGGATG | 180 |
| | 44230725 | CTGATCAAGGTT | |
| 181 | chr27:45473875- | TCATTTTCTTCCTCCCCGTCCCGTCCCGTCCCCTCTGGGACAGA | 181 |
| | 45473877 | CCACAGCAG | |
| 182 | chr27:43319991- | GCCTGTGTCCCCTCAGCCACCGTGATCGTAACGAATTATAGTG | 182 |
| | 43319993 | GCGGAGGATA | |
| 183 | chr18:32818122- | CTGGGCAGGAGCTCGGATCTGAAGTCCGGTGTTGGCGGGGC | 183 |
| | 32818124 | GGGCATCAGCGG | |
| 184 | chr9:6669119- | TCACCCTCCCCAGGGCATGCCAGGGCCGGCCTCAAAGGGGAA | 184 |
| | 6669121 | CGTGAAAGGAA | |
| 185 | chr2:80281700- | GTCCTTTCCCGGCTTCTGGAGGCGCCCGCGTTCCTGGGCTCGG | 185 |
| | 80281702 | GCGCTCCCCG | |
| 186 | chr10:7795521- | GCGGCCTCCGCCCCAACCAGACGCGGCGCGCCCGGGACGCA | 186 |
| | 7795523 | GGGGGCGGTTCT | |
| 187 | chr38:21444492- | TCAGCTCGCCTCTTAGACTCATACAACGCAAGGAACTGAGGAC | 187 |
| | 21444494 | GGGGACATCT | |
| 188 | chr35:16871302- | CTCCCCCGGAGTCAAAGATCTTGACCCGCCTCCTGCCGTCGTG | 188 |
| | 16871304 | CACCACCACG | |
| 189 | chr7:390782-390784 | GCCACCATCTTGTGAGGCGAGCAGGGCGTCGAGGCCGGCGG | 189 |
| | | GTGAGGGGGCGC | |
| 190 | chr38:21444267- | TGTGCGTGCAGGCTGCGGCGAGTGTGCGTGCAGGCTGGGGC | 190 |
| | 21444269 | TGGGGCTGAGTG | |
| 191 | chr30:39153893- | ATACCAGGCGACTGCGCTCAAGGTCTCGGGGTAAAGGCCAGC | |
| | 39153895 | GTCTGGGACGT | 191 |
| 192 | chr3:40933855- | CCCCAGGTAGCTCCCCTTCCTGTCAGCGTGCGGGTGCGTGGG | 192 |
| | 40933857 | GGGCATCCAGG | |
| 193 | chr30:28485837- | GTGTAGGGAGACCCTTCATCCTCAGACGACCCTCAAAACACAT | 193 |
| | 28485839 | ACCTCCCCAA | |
| 194 | chr38:22224460- | GGGCACTTGGCAGGCACTAAGGCAGGCGCTCAGGTGGGTAC | 194 |
| | 22224462 | TTAGTGGGTGCT | |
| 195 | chr27:45470249- | TCACTCGGGCTATGAAACACATCGTGCGCAAAACACCACCCGC | 195 |
| | 45470251 | CGGGCCTGTA | |
| 196 | chr17:1976348- | ACAGCGCGCTTCGCCAGGCCGAGCCCCGCTGCTTTCCAAGACC | 196 |
| | 1976350 | ACCCCCCTCA | |
| 197 | chr38:17700793- | TGTGCATCTGGGGCCCGGTGTGAGTGCGAGACCTGATAGGTG | 197 |
| | 17700795 | TGTGGGGCCCA | |
| 198 | chr9:6669135- | ATGCCAGGGCCGGCCTCAAAGGGGAACGTGAAAGGAACACC | 198 |
| | 6669137 | AGTTATCCCATG | |
| 199 | chr12:72055070- | CCCGCGGCCTTGCCTCGGCCACGCTCCGCCCGAGGGGTCCCC | 199 |
| | 72055072 | GTGGGCTGGGT | |
| 200 | chr14:51535043- | TGCTGGGAGCCCCAGGGGGTGTCTGACGGTCACCCTGGGTTG | 200 |
| | 51535045 | TCACCACCCTA | |

TABLE 2
Summary metrics of the performance per breed of the SVM with linear kernel classifier on the unrelated testing set

| | | | Pos. | Neg. | | | | | | | |
|---|---|---|---|---|---|---|---|---|---|---|---|
| | | | Pred. | Pred. | | | | | Detection. | Detection. | Balanced. |
| | Sensitivity | Specificity | Value | Value | Precision | Recall | F1 | Prevalence | Rate | Prevalence | Accuracy |
| American Fox | 0.67 | 1.00 | 1.00 | 0.99 | 1.00 | 0.67 | 0.80 | 0.03 | 0.02 | 0.02 | 0.83 |
| Hound | | | | | | | | | | | |
| Beagle | 1.00 | 0.97 | 0.94 | 1.00 | 0.94 | 1.00 | 0.97 | 0.30 | 0.30 | 0.32 | 0.99 |
| Brittany | 0.83 | 1.00 | 1.00 | 0.99 | 1.00 | 0.83 | 0.91 | 0.06 | 0.05 | 0.05 | 0.92 |
| Cairn Terrier | 1.00 | 1.00 | 1.00 | 1.00 | 1.00 | 1.00 | 1.00 | 0.04 | 0.04 | 0.04 | 1.00 |
| English Setter | 1.00 | 1.00 | 1.00 | 1.00 | 1.00 | 1.00 | 1.00 | 0.04 | 0.04 | 0.04 | 1.00 |
| French Brittany | 0.00 | 0.99 | 0.00 | 0.00 | 0.00 | 0.00 | 0.00 | 0.00 | 0.00 | 0.01 | 0.00 |
| German Shepherd | 1.00 | 1.00 | 1.00 | 1.00 | 1.00 | 1.00 | 1.00 | 0.01 | 0.01 | 0.01 | 1.00 |
| Dog | | | | | | | | | | | |
| German | 1.00 | 1.00 | 1.00 | 1.00 | 1.00 | 1.00 | 1.00 | 0.01 | 0.01 | 0.01 | 1.00 |
| Shorthaired | | | | | | | | | | | |
| Pointer | | | | | | | | | | | |
| Havanese | 1.00 | 1.00 | 1.00 | 1.00 | 1.00 | 1.00 | 1.00 | 0.01 | 0.01 | 0.01 | 1.00 |
| Labrador | 1.00 | 0.99 | 0.97 | 1.00 | 0.97 | 1.00 | 0.98 | 0.28 | 0.28 | 0.29 | 0.99 |
| Retriever | | | | | | | | | | | |
| Manchester | 0.50 | 1.00 | 1.00 | 0.99 | 1.00 | 0.50 | 0.67 | 0.02 | 0.01 | 0.01 | 0.75 |
| Terrier | | | | | | | | | | | |
| Miniature | 1.00 | 1.00 | 1.00 | 1.00 | 1.00 | 1.00 | 1.00 | 0.08 | 0.08 | 0.08 | 1.00 |
| Schnauzer | | | | | | | | | | | |
| Siberian Husky | 1.00 | 1.00 | 1.00 | 1.00 | 1.00 | 1.00 | 1.00 | 0.03 | 0.03 | 0.03 | 1.00 |
| Smooth Fox Terrier | 0.86 | 1.00 | 1.00 | 0.99 | 1.00 | 0.86 | 0.92 | 0.07 | 0.06 | 0.06 | 0.93 |
| Walker Coonhound | 1.00 | 1.00 | 1.00 | 1.00 | 1.00 | 1.00 | 1.00 | 0.01 | 0.01 | 0.01 | 1.00 |
| Weimaraner | 1.00 | 1.00 | 1.00 | 1.00 | 1.00 | 1.00 | 1.00 | 0.01 | 0.01 | 0.01 | 1.00 |
| Average | 0.87 | 1.00 | 0.93 | 0.93 | 0.93 | 0.87 | 0.89 | 0.06 | 0.06 | 0.06 | 0.90 |

TABLE 3
Coefficients of multinomial logistic regression of breed classifier using 10 methylation sites as features

| | | chr9.31701324- | chr24.22575931- | chr9.6627330- | chr9.5238870- | chr11.65861798- |
|---|---|---|---|---|---|---|
| | Intercept. | 31701326 | 22575933 | 6627332 | 5238872 | 65861800 |
| Beagle | 17.30 | −9.40 | −10.42 | −2.52 | −2.91 | −13.96 |
| Brittany | 6.02 | 0.75 | −9.22 | 0.44 | 11.99 | 2.76 |
| Cairn | −11.96 | −6.68 | 9.62 | −3.25 | 8.71 | −12.38 |
| Terrier | | | | | | |
| English | −3.94 | −11.95 | 10.62 | −11.95 | 6.99 | 0.72 |
| Setter | | | | | | |
| French | −6.30 | −12.58 | 10.18 | 1.96 | 2.36 | 8.83 |
| Brittany | | | | | | |
| German | −3.26 | 4.98 | −4.70 | 8.18 | −0.69 | −3.92 |
| Shepherd | | | | | | |
| Dog | | | | | | |
| German | −0.16 | −6.18 | −2.43 | −6.66 | 8.73 | 8.14 |
| Shorthaired | | | | | | |
| Pointer | | | | | | |
| Havanese | −12.78 | 0.83 | 14.04 | 7.02 | −1.65 | 3.81 |
| Labrador | −4.99 | −0.24 | 19.46 | 11.41 | −6.76 | 28.45 |
| Retriever | | | | | | |
| Manchester | 3.47 | 7.53 | −9.86 | −0.81 | 1.34 | −7.29 |
| Terrier | | | | | | |
| Miniature | −18.70 | 2.40 | 6.27 | 14.57 | −3.94 | −11.29 |
| Schnauzer | | | | | | |
| Siberian | 5.51 | −10.58 | 0.33 | −5.28 | 12.69 | 4.90 |
| Husky | | | | | | |
| Smooth | −2.38 | 7.70 | −15.28 | 10.78 | 9.42 | −16.01 |
| Fox | | | | | | |
| Terrier | | | | | | |
| Walker | −5.93 | 10.07 | −24.42 | 3.63 | 3.22 | 15.74 |
| Coonhound | | | | | | |
| Weimaraner | 12.57 | −6.40 | 1.21 | −3.37 | 12.12 | −8.13 |

| | chr3.74353518- | chr5.63708131- | chr3.63619987- | chr5.15661221- | chr29.39142945- |
|---|---|---|---|---|---|
| | 74353520 | 63708133 | 63619989 | 15661223 | 39142947 |
| Beagle | 0.25 | 6.53 | −1.59 | 2.54 | −3.55 |
| Brittany | −0.48 | −2.80 | −5.22 | −7.48 | −0.10 |
| Cairn | 5.87 | 7.81 | −5.76 | 4.48 | −3.02 |
| Terrier | | | | | |
| English | 9.55 | −0.27 | −7.75 | −1.45 | 0.01 |
| Setter | | | | | |
| French | 4.08 | −2.63 | 8.43 | −2.30 | −2.02 |
| Brittany | | | | | |
| German | 2.90 | 5.18 | −14.12 | −3.38 | −0.46 |
| Shepherd | | | | | |
| Dog | | | | | |
| German | −6.69 | −12.83 | −5.77 | 14.16 | 5.99 |
| Shorthaired | | | | | |
| Pointer | | | | | |
| Havanese | 3.33 | −5.38 | 6.62 | −8.54 | −0.55 |
| Labrador | 5.43 | −7.07 | −9.75 | −1.55 | −0.42 |
| Retriever | | | | | |
| Manchester | 4.07 | −6.54 | −10.72 | 1.07 | 6.46 |
| Terrier | | | | | |
| Miniature | 2.12 | 6.45 | −10.29 | 2.18 | 7.38 |
| Schnauzer | | | | | |
| Siberian | −0.14 | −14.05 | 3.01 | 0.63 | 3.87 |
| Husky | | | | | |
| Smooth | −0.43 | −4.39 | 8.15 | −5.72 | −11.01 |
| Fox | | | | | |
| Terrier | | | | | |
| Walker | −0.32 | 9.58 | 6.45 | 0.95 | −8.96 |
| Coonhound | | | | | |
| Weimaraner | −1.92 | −12.13 | −3.52 | −3.06 | −1.40 |

TABLE 4
F1 training cross-validation on different numbers of sites

| Number of sites | F1_train_cross_validated |
|---|---|
| 5 | 0.342923932 |
| 10 | 0.558679764 |
| 20 | 0.618192168 |
| 25 | 0.678316505 |
| 50 | 0.735451126 |
| 100 | 0.754503632 |
| 150 | 0.760859564 |
| 200 | 0.768644068 |

1. A method of determining the contribution of a dog breed to a test dog genome, comprising:
a) providing a DNA methylation profile from a sample obtained from the test dog; and
b) determining the contribution of a dog breed to the test dog genome by comparing at least part of the DNA methylation profile of the test dog to reference DNA methylation profiles from different dog breeds.

2. A method for selecting a dietary, pharmacological, or lifestyle regime for a test dog, the method comprising:
a) providing a DNA methylation profile from a sample obtained from the test dog;
b) determining the contribution of a dog breed to the test dog genome by comparing at least part of the DNA methylation profile of the test dog to reference DNA methylation profiles from different dog breeds; and
c) selecting a suitable dietary, pharmacological, or lifestyle regime for the test dog based on the contribution of a dog breed to the test dog genome determined in step b).

3. A method for preventing or reducing the risk of a test dog developing a disease; the method comprising:
a) providing a DNA methylation profile from a sample obtained from the test dog;
b) determining the contribution of a dog breed to the test dog genome by comparing at least part of the DNA methylation profile of the test dog to reference DNA methylation profiles from different dog breeds; wherein at least one dog breed contributing to the test dog genome is associated with a propensity to develop a disease; and
c) selecting a dietary, pharmacological, or lifestyle regime for the test dog based on the contribution of the at least one dog breed to the test dog genome determined in step b);
wherein the dietary, pharmacological, or lifestyle regime prevents or reduces the risk of the test dog developing the disease.

4. The method according to claim 1, wherein step a) comprises determining a DNA methylation profile from a sample obtained from the test dog.

5. The method according to claim 4, wherein DNA methylation is determined according to a method which comprises: (i) one or more of the following steps: (a) treating the sample DNA with APOBEC or using bisulfite conversion to deaminate unmethylated cytosines; (b) a capture-based enrichment; and/or (c) high throughput sequencing or arrays; or (ii) de novo sequencing.

6. The method according to claim 1, wherein the contribution of at least two dog breeds to the test dog genome is determined.

7. The method according to claim 1, wherein the DNA methylation profile of the test dog is compared to reference DNA methylation profiles from different dog breeds using machine learning.

8. The method according to claim 1, wherein the reference DNA methylation profiles comprise DNA methylation profiles from at least 2, at least 4, at least 10, at least 20, at least 40, or at least 80 dog breeds.

9. The method according to claim 1, wherein the DNA methylation profile comprises at least one population specific DNA methylation marker.

10. The method according to claim 1, wherein the contributions of dog breeds to the test dog genome is used to distinguish between two of more genetically related dog breeds.

11. The method according to claim 1, wherein the contributions of dog breeds to the test dog genome is used to classify the test dog as: (i) an American Kennel Club registered breed; (ii) a genetic breed clade; (iii) a breed size; and/or (iv) a robust or athletic breed.

12. The method according to claim 11, wherein the genetic breed clade is selected from Wild, Basenji, Asian Spitz, Asian Toy, Nordic Spitz, Schnauzer, Small Spitz, Toy Spitz, Hungarian, Poodle, American Terrier, American Toy, Pinscher, Terrier, New World, Mediterranean, Scent Hound, Retriever, Pointer Setter, Continental Herder, UK Rural, Drover, Alpine, and European Mastiff.

13. The method according to claim 2, wherein the dietary, pharmacological, or lifestyle regime is a dietary intervention.

14. The method according to claim 13, wherein the dietary intervention is a calorie-restricted diet, a senior diet or a low protein diet.

15. The method according to claim 1, wherein the sample is a blood sample.

16. The method according to claim 1, wherein the DNA methylation profile comprises at least 10 methylation sites.

17. The method according to claim 1, wherein the DNA methylation profile comprises at least one methylation site as listed in Table 1.

18. The method according to claim 17, wherein the DNA methylation profile comprises at least 2, at least 5, at least 10, at least 20, at least 50, at least 100, at least 150, at least 175 or each of the methylation sites as listed in Table 1.

19. The method according to claim 3, wherein the disease is associated with a morbidity or predicted morbidity of (i) a tissue; (ii) an organ; or (iii) a physiological system, such as the immune, gastrointestinal, urinary, muscular, cardiovascular, and/or neurological system.

20. The method according to claim 19, further comprising applying to the dog a dietary, pharmacological, or lifestyle regime which is suitable for improving the morbidity or predicted morbidity of the tissue; organ; or physiological system identified in claim 19.

21. A computer-readable medium comprising instructions that when executed cause one or more processors to perform the method of claim 1.

22. A computer system for determining the contributions of a dog breed to a test dog genome, the computer system programmed to compare at least part of a DNA methylation profile obtained from the test dog to reference DNA methylation profiles from different dog breeds.

23. A computer system for selecting a dietary, pharmacological, or lifestyle regime for a test dog, the computer system programmed to perform the steps of:
a) determining the contributions of a dog breed to the test dog genome by comparing at least part of a DNA methylation profile obtained from the test dog to reference DNA methylation profiles from different dog breeds; and
b) selecting a suitable dietary, pharmacological, or lifestyle regime for the test dog based on the contribution of the dog breed to the test dog genome determined in step a).

24. A computer program product comprising computer implementable instructions for causing a programmable computer to determine the contributions of a dog breed to a test dog genome by comparing at least part of a DNA methylation profile obtained from a test dog to reference DNA methylation profiles from different dog breeds.

25. A computer program product comprising computer implementable instructions for causing a programmable computer to select a dietary, pharmacological, or lifestyle regime for a test dog by a) determining the contributions of a dog breed to the test dog genome by comparing at least part of a DNA methylation profile obtained from the test dog to reference DNA methylation profiles from different dog breeds; and b) selecting a suitable dietary, pharmacological, or lifestyle regime for the test dog based on the contributions of the dog breed to the test dog genome determined in step a).


SEQUENCE LISTING (200 sequences)

SEQ ID NO: 1 - DNA, 53 bp, Canis lupus
  source 1..53: mol_type = genomic DNA; note = canis familiaris
catctacgcaaatggtcttcccgttacgtggattttcattggctcatcaccct

SEQ ID NO: 2 - DNA, 53 bp, Canis lupus
  source 1..53: mol_type = genomic DNA; note = canis familiaris
agcgcctctgcacctgtttccctgaccggtatgcctgactcccatggtgggct

SEQ ID NO: 3 - DNA, 53 bp, Canis lupus
  source 1..53: mol_type = genomic DNA; note = canis familiaris
tgagggcaccctcgctcgaactcagccgggggtgtccctggagggccaggctc

SEQ ID NO: 4 - DNA, 53 bp, Canis lupus
  source 1..53: mol_type = genomic DNA; note = canis familiaris
aacacttgcacatccacgagcaggggcgcgctgccattgtttggaatctggat

SEQ ID NO: 5 - DNA, 53 bp, Canis lupus
  source 1..53: mol_type = genomic DNA; note = canis familiaris
gagtgcagccaaataagcaattggagcgggaaggactgctccctgagctacgt

SEQ ID NO: 6 - DNA, 53 bp, Canis lupus
  source 1..53: mol_type = genomic DNA; note = canis familiaris
cagaagttagtaagaacaggtaaggccgtctccacgtcatcgtttctgtacat

SEQ ID NO: 7 - DNA, 53 bp, Canis lupus
  source 1..53: mol_type = genomic DNA; note = canis familiaris
caccacagcagcctgagcagccaaggcgcctgccgccaccagcagcatgacgg

SEQ ID NO: 8 - DNA, 53 bp, Canis lupus
  source 1..53: mol_type = genomic DNA; note = canis familiaris
ctggcgatgggcctcccctggcagcccgctggccttcactatgcacaggacca

SEQ ID NO: 9 - DNA, 53 bp, Canis lupus
  source 1..53: mol_type = genomic DNA; note = canis familiaris
ccaagaccacacagtaaattagcgtccgattccgaacaaatccaggtcttcgg

SEQ ID NO: 10 - DNA, 53 bp, Canis lupus
  source 1..53: mol_type = genomic DNA; note = canis familiaris
aggaacgactcagaaagcacagcccacggtgcaccccgcggcgcccgccctca

SEQ ID NO: 11 - DNA, 53 bp, Canis lupus
  source 1..53: mol_type = genomic DNA; note = canis familiaris
ccaccctttcagagggaccctcctagcgccctgtaaagtgctctcctccctca

SEQ ID NO: 12 - DNA, 53 bp, Canis lupus
  source 1..53: mol_type = genomic DNA; note = canis familiaris
caggtccacagtgtgagcacggtgaccgaggacgtgcggacaccttggtgagc

SEQ ID NO: 13 - DNA, 53 bp, Canis lupus
  source 1..53: mol_type = genomic DNA; note = canis familiaris
tcgcctggcaaagacccttggcctcccgagatttcagggtccctgggggggcc

SEQ ID NO: 14 - DNA, 53 bp, Canis lupus
  source 1..53: mol_type = genomic DNA; note = canis familiaris
gggagggcctcgcccccgcagcctctcgtcccgcggtctccccctccgcactt

SEQ ID NO: 15 - DNA, 53 bp, Canis lupus
  source 1..53: mol_type = genomic DNA; note = canis familiaris
gtgccaccgccgctgctcctgcctcccggcccgctgcacggtccaggtcttcc

SEQ ID NO: 16 - DNA, 53 bp, Canis lupus
  source 1..53: mol_type = genomic DNA; note = canis familiaris
gcagcgtttcgttgaaaagaaaggtccgtgggggaagtggggaccccggttag

SEQ ID NO: 17 - DNA, 53 bp, Canis lupus
  source 1..53: mol_type = genomic DNA; note = canis familiaris
tttccccgccaggcccccggctaatgcgtcccccggcattcgccagcgctcat

SEQ ID NO: 18 - DNA, 53 bp, Canis lupus
  source 1..53: mol_type = genomic DNA; note = canis familiaris
gctctgacgtggaagacaggtcaacacgagcgtcatccaggtgtacaactgta

SEQ ID NO: 19 - DNA, 53 bp, Canis lupus
  source 1..53: mol_type = genomic DNA; note = canis familiaris
cccagaggacgccaggctgacggggtcgaggccaaggccgctacccttgaggc

SEQ ID NO: 20 - DNA, 53 bp, Canis lupus
  source 1..53: mol_type = genomic DNA; note = canis familiaris
agtgccgacagccacggggctgggggcgagggacgtctatccagggggttcct

SEQ ID NO: 21 - DNA, 53 bp, Canis lupus
  source 1..53: mol_type = genomic DNA; note = canis familiaris
agtcacgctgccaacgaggctaggcccgaccgagtcgcccggccacggcgctg

SEQ ID NO: 22 - DNA, 53 bp, Canis lupus
  source 1..53: mol_type = genomic DNA; note = canis familiaris
atgagcgggtcaaagagcgcgcacgtcgctctggctcccccgcaacaccgcca

SEQ ID NO: 23 - DNA, 53 bp, Canis lupus
  source 1..53: mol_type = genomic DNA; note = canis familiaris
gggaccgctgtggacgtttccgtgggcgtccgcctgcaacgtccctcggccct

SEQ ID NO: 24 - DNA, 53 bp, Canis lupus
  source 1..53: mol_type = genomic DNA; note = canis familiaris
agcgccatcagtaaaccccacagctgcggtcgcacacagcagagcaggccagg

SEQ ID NO: 25 - DNA, 53 bp, Canis lupus
  source 1..53: mol_type = genomic DNA; note = canis familiaris
ccctggcccctccatgcctgggaccacgcaaccacgtgaggaaacgttttgtt

SEQ ID NO: 26 - DNA, 53 bp, Canis lupus
  source 1..53: mol_type = genomic DNA; note = canis familiaris
gtaatagaaggtgaaattgatgtggacggtgccacccttgggggcggagaggt

SEQ ID NO: 27 - DNA, 53 bp, Canis lupus
  source 1..53: mol_type = genomic DNA; note = canis familiaris
gggcctccaagccctgtttccatgtccggtacctgctcaaggcacaggcgacc

SEQ ID NO: 28 - DNA, 53 bp, Canis lupus
  source 1..53: mol_type = genomic DNA; note = canis familiaris
caaggtcacggacagcctgcgagactcgtcaaggtcacggacagcctgcgtga

SEQ ID NO: 29 - DNA, 53 bp, Canis lupus
  source 1..53: mol_type = genomic DNA; note = canis familiaris
cacaggtgcttagtacagggcccttgcgccccaccttgctgctgcgaacaaaa

SEQ ID NO: 30 - DNA, 53 bp, Canis lupus
  source 1..53: mol_type = genomic DNA; note = canis familiaris
ggccagctgatggggtccccctgggacggagattctcttgccaacaagtcaag

SEQ ID NO: 31 - DNA, 53 bp, Canis lupus
  source 1..53: mol_type = genomic DNA; note = canis familiaris
gaagtctgatgatgggagacgggcagcgactgcagcgagacacagggcatgtc

SEQ ID NO: 32 - DNA, 53 bp, Canis lupus
  source 1..53: mol_type = genomic DNA; note = canis familiaris
ctgcagcccagaggacgccaggctgacggggtcgaggccaaggccgctaccct

SEQ ID NO: 33 - DNA, 53 bp, Canis lupus
  source 1..53: mol_type = genomic DNA; note = canis familiaris
cgggcgcgcccaggccggggccccggcggccgaccatggtgctgccgcccccg

SEQ ID NO: 34 - DNA, 53 bp, Canis lupus
  source 1..53: mol_type = genomic DNA; note = canis familiaris
tgccccacgccccactgggccccctccgggcccagaaatgcggcctcccctcc

SEQ ID NO: 35 - DNA, 53 bp, Canis lupus
  source 1..53: mol_type = genomic DNA; note = canis familiaris
tcactcagtttcctattcatcgttcccgctctccacccttcctttctgcctct

SEQ ID NO: 36 - DNA, 53 bp, Canis lupus
  source 1..53: mol_type = genomic DNA; note = canis familiaris
agatcctcccgcctctccgcagggcccgcagcaccgctcccgttgggggtgca

SEQ ID NO: 37 - DNA, 53 bp, Canis lupus
  source 1..53: mol_type = genomic DNA; note = canis familiaris
cgatgggcccagaggctccctccctacgctcccacaacgctctgtaaacacct

SEQ ID NO: 38 - DNA, 53 bp, Canis lupus
  source 1..53: mol_type = genomic DNA; note = canis familiaris
tcacacaccagccctccggggaaggccgcggagccgtcgcgccaccagcggtc

SEQ ID NO: 39 - DNA, 53 bp, Canis lupus
  source 1..53: mol_type = genomic DNA; note = canis familiaris
agctggcaagtagcatggggagcacccgaatctagtcacctaacttcaaatcc

SEQ ID NO: 40 - DNA, 53 bp, Canis lupus
  source 1..53: mol_type = genomic DNA; note = canis familiaris
cacaccccactcggcaccaccacccgcgggttctcccggcagtgcctgcattg

SEQ ID NO: 41 - DNA, 53 bp, Canis lupus
  source 1..53: mol_type = genomic DNA; note = canis familiaris
taggagagagtggatgccttatctttcgtgatttccgccaagacaattatggg

SEQ ID NO: 42 - DNA, 53 bp, Canis lupus
  source 1..53: mol_type = genomic DNA; note = canis familiaris
aagggatgtgacttgcctacagagcacgatattggccaggtcaggtctccctc

SEQ ID NO: 43 - DNA, 53 bp, Canis lupus
  source 1..53: mol_type = genomic DNA; note = canis familiaris
ggctggaagagtgtcctccccgcgagcggggctcctggcggggggcgggggct

SEQ ID NO: 44 - DNA, 53 bp, Canis lupus
  source 1..53: mol_type = genomic DNA; note = canis familiaris
ccgaggtgtcaggtcgtagctggggacgggagcgcggctcggcacctggtggg

SEQ ID NO: 45 - DNA, 53 bp, Canis lupus
  source 1..53: mol_type = genomic DNA; note = canis familiaris
tgaaaacccagcctgcattccccctgcgccatgtccctgtccccagccctcca

SEQ ID NO: 46 - DNA, 53 bp, Canis lupus
  source 1..53: mol_type = genomic DNA; note = canis familiaris
agaaaggcacgtgactgtgcatgcgccgacgggaggcgcgtgggcagagaaag

SEQ ID NO: 47 - DNA, 53 bp, Canis lupus
  source 1..53: mol_type = genomic DNA; note = canis familiaris
ggggactcaggtgggcactcggcgggcgctcaggcgggcactcggtgggcact

SEQ ID NO: 48 - DNA, 53 bp, Canis lupus
  source 1..53: mol_type = genomic DNA; note = canis familiaris
tctaagacccctgtcagcgctgggcccgtgcgcagggaggcgcggagcagcag

SEQ ID NO: 49 - DNA, 53 bp, Canis lupus
  source 1..53: mol_type = genomic DNA; note = canis familiaris
ctcctgggcctcatgttcaggggacacggcacaggggtctgggtccgtgggcc

SEQ ID NO: 50 - DNA, 53 bp, Canis lupus
  source 1..53: mol_type = genomic DNA; note = canis familiaris
ccccaccagcttcaccatccataacccgtctcagggccatccaccctgtcctt

SEQ ID NO: 51 - DNA, 53 bp, Canis lupus
  source 1..53: mol_type = genomic DNA; note = canis familiaris
gcggggctcccctcctcccccgatcccggcctccagttatctgggtgaggcag

SEQ ID NO: 52 - DNA, 53 bp, Canis lupus
  source 1..53: mol_type = genomic DNA; note = canis familiaris
catgaaccacggtgagttggtgggcccgggtgtggggttgggggcaggtagca

SEQ ID NO: 53 - DNA, 53 bp, Canis lupus
  source 1..53: mol_type = genomic DNA; note = canis familiaris
aggccaccctgtacctagaggggcgccggggggggctcgcggggtgcagggca

SEQ ID NO: 54 - DNA, 53 bp, Canis lupus
  source 1..53: mol_type = genomic DNA; note = canis familiaris
aggtgctgcggacgtggtgcgggctgcgcgacgtgtaccaggtgagccgggcg

SEQ ID NO: 55 - DNA, 53 bp, Canis lupus
  source 1..53: mol_type = genomic DNA; note = canis familiaris
tgaagcggacacaccacgtgccagagcggcccatcccagtggcacagcccagg

SEQ ID NO: 56 - DNA, 53 bp, Canis lupus
  source 1..53: mol_type = genomic DNA; note = canis familiaris
gctcactggcacttggcgcctccacgcggggcaggctgaccctgggcagcgcc

SEQ ID NO: 57 - DNA, 53 bp, Canis lupus
  source 1..53: mol_type = genomic DNA; note = canis familiaris
cccaggactgtgaaggcctcatcgtccgttcggccaccaaggtgactgctgac

SEQ ID NO: 58 - DNA, 53 bp, Canis lupus
  source 1..53: mol_type = genomic DNA; note = canis familiaris
cgcatgaaaaccgacacacctggccacgcgcacttaagtttctcagcttctct

SEQ ID NO: 59 - DNA, 53 bp, Canis lupus
  source 1..53: mol_type = genomic DNA; note = canis familiaris
caccccactggacaatgaggtctgctcggctaaaaatatccctgagccactaa

SEQ ID NO: 60 - DNA, 53 bp, Canis lupus
  source 1..53: mol_type = genomic DNA; note = canis familiaris
tcctcaccgcaggctcctacctaactcgatcccgtcctcaccgcaggctccta

SEQ ID NO: 61 - DNA, 53 bp, Canis lupus
  source 1..53: mol_type = genomic DNA; note = canis familiaris
acacggtgaacgcctggtcaggactccggggacccagccctcttcattagggg

SEQ ID NO: 62 - DNA, 53 bp, Canis lupus
  source 1..53: mol_type = genomic DNA; note = canis familiaris
tcatccccatttacagatgagcacaccgattctcccagcagctgtcacttgct

SEQ ID NO: 63 - DNA, 53 bp, Canis lupus
  source 1..53: mol_type = genomic DNA; note = canis familiaris
caagaggcccgccctgcgctggggcgcggccgggagctcagacctggctcacc

SEQ ID NO: 64 - DNA, 53 bp, Canis lupus
  source 1..53: mol_type = genomic DNA; note = canis familiaris
ctcgacgctgctgtccccgaaggccgcggccccccagaggtacgcatcgctga

SEQ ID NO: 65 - DNA, 53 bp, Canis lupus
  source 1..53: mol_type = genomic DNA; note = canis familiaris
ggcagctaggtgcccaccgccacctccgctgctgcccatgcaggtccctgggc

SEQ ID NO: 66 - DNA, 53 bp, Canis lupus
  source 1..53: mol_type = genomic DNA; note = canis familiaris
atttttaccagagatattcccattaacgttaatttgtagaccaagtctgtcta

SEQ ID NO: 67 - DNA, 53 bp, Canis lupus
  source 1..53: mol_type = genomic DNA; note = canis familiaris
tggtgtctgcacttactgtgatctctcgatttcatgcttcatcaaatattcag

SEQ ID NO: 68 - DNA, 53 bp, Canis lupus
  source 1..53: mol_type = genomic DNA; note = canis familiaris
tagctgggagactctcagcgcaagtccgaagggccggcttcttgtgcccctcc

SEQ ID NO: 69 - DNA, 53 bp, Canis lupus
  source 1..53: mol_type = genomic DNA; note = canis familiaris
aagagtcccctggggcctggcgactccgagtgagtaggacctgtggttgtcac

SEQ ID NO: 70 - DNA, 53 bp, Canis lupus
  source 1..53: mol_type = genomic DNA; note = canis familiaris
tggagaagctcagctcagcccagcctcggcctccttgctggtctgactggtaa

SEQ ID NO: 71 - DNA, 53 bp, Canis lupus
  source 1..53: mol_type = genomic DNA; note = canis familiaris
aagccctccgatccgcccgatcggcccgggctccccgtcctcgcacctccgca

SEQ ID NO: 72 - DNA, 53 bp, Canis lupus
  source 1..53: mol_type = genomic DNA; note = canis familiaris
gaggtgcttctgaaccccagtctctgcgcttcctgcagctagctcctcgcgtt

SEQ ID NO: 73 - DNA, 53 bp, Canis lupus
  source 1..53: mol_type = genomic DNA; note = canis familiaris
gggtggtgtgcagctgtgtcacctcacgtcctggccacctgcccagagctggc

SEQ ID NO: 74 - DNA, 53 bp, Canis lupus
  source 1..53: mol_type = genomic DNA; note = canis familiaris
cttgggaggaggcccagcacccatcccgcatcttggtcctttgggattcaacc

SEQ ID NO: 75 - DNA, 53 bp, Canis lupus
  source 1..53: mol_type = genomic DNA; note = canis familiaris
ttggcaatcatggcctacgatcgctacgtggccatctgccacccgctgcacta

SEQ ID NO: 76 - DNA, 53 bp, Canis lupus
  source 1..53: mol_type = genomic DNA; note = canis familiaris
cccggaacctgcagccgggaaaggcccgggtgtccaggaaagccctgtgcctc

SEQ ID NO: 77 - DNA, 53 bp, Canis lupus
  source 1..53: mol_type = genomic DNA; note = canis familiaris
tggcccaagctaccagcctctcccaccgagactactggtctcccactgctctc

SEQ ID NO: 78 - DNA, 53 bp, Canis lupus
  source 1..53: mol_type = genomic DNA; note = canis familiaris
tggtgcagctgctgctgcagcgctggcgggacccggagtcaggcctggactca

SEQ ID NO: 79 - DNA, 53 bp, Canis lupus
  source 1..53: mol_type = genomic DNA; note = canis familiaris
gctccgggcctccaggccgcggcctccgtggggctggtgcgcgctgcagccgc

SEQ ID NO: 80 - DNA, 53 bp, Canis lupus
  source 1..53: mol_type = genomic DNA; note = canis familiaris
ccaaagctagggtagcagtcgctagacgctgccgaaaccccaggcctgacgtt

SEQ ID NO: 81 - DNA, 53 bp, Canis lupus
  source 1..53: mol_type = genomic DNA; note = canis familiaris
aagtctccaccccgcagtggactatccgcctagttttgtgccagtcccaaagg

SEQ ID NO: 82 - DNA, 53 bp, Canis lupus
  source 1..53: mol_type = genomic DNA; note = canis familiaris
agcaaagggtgttatttggggcatgccgggatcagcactgctttaacccgagg

SEQ ID NO: 83 - DNA, 53 bp, Canis lupus
  source 1..53: mol_type = genomic DNA; note = canis familiaris
aggggtgcaggacgtgagcaagaaaccgcgctctcctgcaaggctctgtttaa

SEQ ID NO: 84 - DNA, 53 bp, Canis lupus
  source 1..53: mol_type = genomic DNA; note = canis familiaris
tcctcaccgcaggctcctacctaactcgatcctgtcctcactgcaggctccaa

SEQ ID NO: 85 - DNA, 53 bp, Canis lupus; identical to SEQ ID NO: 75
  source 1..53: mol_type = genomic DNA; note = canis familiaris

SEQ ID NO: 86 - DNA, 53 bp, Canis lupus
  source 1..53: mol_type = genomic DNA; note = canis familiaris
cgtctgctacaggccgggcaccgccacggatttaatagggtcgtgatttttag

SEQ ID NO: 87 - DNA, 53 bp, Canis lupus
  source 1..53: mol_type = genomic DNA; note = canis familiaris
cggacccctcggcagggatgcgcatgcgtcctgtccactggccgcaccgtgga

SEQ ID NO: 88 - DNA, 53 bp, Canis lupus
  source 1..53: mol_type = genomic DNA; note = canis familiaris
actcacacctgtcgaacacctcaatccgtgctccttcctctgtgtcaagctgc

SEQ ID NO: 89 - DNA, 53 bp, Canis lupus
  source 1..53: mol_type = genomic DNA; note = canis familiaris
ctagaaaagagcagatcctgacctaccgtggtcacactagatgctgggcacat

SEQ ID NO: 90 - DNA, 53 bp, Canis lupus
  source 1..53: mol_type = genomic DNA; note = canis familiaris
tggtggggtcgcactgcgggctggcgcgggtccctccagcccatccctccccg

SEQ ID NO: 91 - DNA, 53 bp, Canis lupus
  source 1..53: mol_type = genomic DNA; note = canis familiaris
aggcagggacagacagggggttcgctcgggggctccttcctctagggacagag

SEQ ID NO: 92 - DNA, 53 bp, Canis lupus
  source 1..53: mol_type = genomic DNA; note = canis familiaris
ggtcacagacataggctgccagtagacgcgatcaataggtgctggttgaatat

SEQ ID NO: 93 - DNA, 53 bp, Canis lupus
  source 1..53: mol_type = genomic DNA; note = canis familiaris
aaggccggccgcgccgcactctggggcgcccccctgcacccagcagacaccca

SEQ ID NO: 94 - DNA, 53 bp, Canis lupus
  source 1..53: mol_type = genomic DNA; note = canis familiaris
gggcctgtgagtgtgcatggtcgggtcggggctgagcgaactaagccttgggg

SEQ ID NO: 95 - DNA, 53 bp, Canis lupus
  source 1..53: mol_type = genomic DNA; note = canis familiaris
caccacggccaacagcctgggctgcacgtcacttgcctggccgccatggcagt

SEQ ID NO: 96 - DNA, 53 bp, Canis lupus
  source 1..53: mol_type = genomic DNA; note = canis familiaris
tctctgacatgtctgtctcctccattcgttcacacatttcctgagctcccagc

SEQ ID NO: 97 - DNA, 53 bp, Canis lupus
  source 1..53: mol_type = genomic DNA; note = canis familiaris
cttcctggtcaggccctgtcccctgtcgcttctccagaaaagcaagcaccaaa

SEQ ID NO: 98 - DNA, 53 bp, Canis lupus
  source 1..53: mol_type = genomic DNA; note = canis familiaris
ttagggcactgcatgcgagcccggggcgggggggggcacggcagccaccgggg

SEQ ID NO: 99 - DNA, 53 bp, Canis lupus
  source 1..53: mol_type = genomic DNA; note = canis familiaris
tttccatgggctgtttttattttctccgcattttctccctactcttcttcttc

SEQ ID NO: 100 - DNA, 53 bp, Canis lupus
  source 1..53: mol_type = genomic DNA; note = canis familiaris
ctgtctccaggcttttcctcacctctcgccatatcctgctgattctgcttcct

SEQ ID NO: 101 - DNA, 53 bp, Canis lupus
  source 1..53: mol_type = genomic DNA; note = canis familiaris
cagggggagcgaggaggagtggccagcgagactctccagaccaacgacgtgct

SEQ ID NO: 102 - DNA, 53 bp, Canis lupus
  source 1..53: mol_type = genomic DNA; note = canis familiaris
gtgcaatgaaccggttcaatccaccgcgcttgttgtggtttgaaggtgagtgc

SEQ ID NO: 103 - DNA, 53 bp, Canis lupus
  source 1..53: mol_type = genomic DNA; note = canis familiaris
agcacactgccccagggcaggtgccgcgaggataccgggcttccagaagagct

SEQ ID NO: 104 - DNA, 53 bp, Canis lupus
  source 1..53: mol_type = genomic DNA; note = canis familiaris
ccgggggctctgggcctcctgagccccgcggggtgtgtgcgtgcgtgtgcacg

SEQ ID NO: 105 - DNA, 53 bp, Canis lupus
  source 1..53: mol_type = genomic DNA; note = canis familiaris
tcagcagcttccacttccacccctgccgggagctgcttgtgcagtttgttggg

SEQ ID NO: 106 - DNA, 53 bp, Canis lupus
  source 1..53: mol_type = genomic DNA; note = canis familiaris
agccggctcctctgctctgactcagccgcagccctgctgcctctggcccagca

SEQ ID NO: 107 - DNA, 53 bp, Canis lupus
  source 1..53: mol_type = genomic DNA; note = canis familiaris
ctagtgccagactgcagcccagaggacgccaggctgacggggtcgaggccaag

SEQ ID NO: 108 - DNA, 53 bp, Canis lupus
  source 1..53: mol_type = genomic DNA; note = canis familiaris
tgggcctagaggccacaactgcttctcgaggtcactgaaggccccccacatcc

SEQ ID NO: 109 - DNA, 53 bp, Canis lupus
  source 1..53: mol_type = genomic DNA; note = canis familiaris
gacttggtaacctgtctcccttcacccgcgctgcctcctcagatccacgaaac

SEQ ID NO: 110 - DNA, 53 bp, Canis lupus
  source 1..53: mol_type = genomic DNA; note = canis familiaris
tgatttatggatgttttggactaacccgccctacatagcttcggaactagatc

SEQ ID NO: 111 - DNA, 53 bp, Canis lupus
  source 1..53: mol_type = genomic DNA; note = canis familiaris
ggggcgtggcttctttccttctactgcgtcttttgttatctgcaatcttggac

SEQ ID NO: 112 - DNA, 53 bp, Canis lupus
  source 1..53: mol_type = genomic DNA; note = canis familiaris
gactgcccactgttaggcggcctctccgggcctggcgctgaggggtgcttgcc

SEQ ID NO: 113 - DNA, 53 bp, Canis lupus
  source 1..53: mol_type = genomic DNA; note = canis familiaris
gccgcctgccgtccctccgtctccggcgcctctacctgtgttctcgggtcacc

SEQ ID NO: 114 - DNA, 53 bp, Canis lupus
  source 1..53: mol_type = genomic DNA; note = canis familiaris
cagccttttcaaatcccagagatacgcgcgcatgagcagaccacccggtttgt

SEQ ID NO: 115 - DNA, 53 bp, Canis lupus
  source 1..53: mol_type = genomic DNA; note = canis familiaris
cggcgcgacccccgcgcgactcctcacggctgcggtgctctcgcccttcacag

SEQ ID NO: 116 - DNA, 53 bp, Canis lupus
  source 1..53: mol_type = genomic DNA; note = canis familiaris
tgtttccggaaggcagccagagggagcgctcagggcctttgcacctgctcgaa

SEQ ID NO: 117 - DNA, 53 bp, Canis lupus
  source 1..53: mol_type = genomic DNA; note = canis familiaris
tccggcccatcctctggcttgacttccgtcgtggtgcttaattctccccgcaa

SEQ ID NO: 118 - DNA, 53 bp, Canis lupus
  source 1..53: mol_type = genomic DNA; note = canis familiaris
agtggttctgtgacggcgggaggcttcgggacgctttcctgtgctccgtgagt

SEQ ID NO: 119 - DNA, 53 bp, Canis lupus
  source 1..53: mol_type = genomic DNA; note = canis familiaris
ccagaacttggggtcagtcccttgcccggaggccccacctccgccccccaagg

SEQ ID NO: 120 - DNA, 53 bp, Canis lupus
  source 1..53: mol_type = genomic DNA; note = canis familiaris
agttcacagaatatcgccgacttatccggctaattcccagacccacacctcaa

SEQ ID NO: 121 - DNA, 53 bp, Canis lupus
  source 1..53: mol_type = genomic DNA; note = canis familiaris
ctcagccacgccgcctccttcggggccggggccctctggctgcgcgggccccg

SEQ ID NO: 122 - DNA, 53 bp, Canis lupus
  source 1..53: mol_type = genomic DNA; note = canis familiaris
taggcaaagggggaaagggccgtaatcgacctggctcttctccactggatggg

SEQ ID NO: 123 - DNA, 53 bp, Canis lupus
  source 1..53: mol_type = genomic DNA; note = canis familiaris
aagggattccacctgcttacaggccccggaagctcccctggaacccacatcac

SEQ ID NO: 124 - DNA, 53 bp, Canis lupus
  source 1..53: mol_type = genomic DNA; note = canis familiaris
tcgagggaaagtgagctgctcctggccggcccgccctctccctggccctggca

SEQ ID NO: 125 - DNA, 53 bp, Canis lupus
  source 1..53: mol_type = genomic DNA; note = canis familiaris
agttgtttaccaaggcctggttctcacgtgtgttctggccttcgccctcggcc

SEQ ID NO: 126 - DNA, 53 bp, Canis lupus
  source 1..53: mol_type = genomic DNA; note = canis familiaris
gctctagggagagaagagcaagctaccgacccaggaatcctacagatcagacc

SEQ ID NO: 127 - DNA, 53 bp, Canis lupus
  source 1..53: mol_type = genomic DNA; note = canis familiaris
actgcaggctccaactgactcgatcccgtcctcaccgcaggctcctacctaac

SEQ ID NO: 128 - DNA, 53 bp, Canis lupus
  source 1..53: mol_type = genomic DNA; note = canis familiaris
aggggggccaggaggttcttgttgaccgtgacctgatggatgccccccggagg

SEQ ID NO: 129 - DNA, 53 bp, Canis lupus
  source 1..53: mol_type = genomic DNA; note = canis familiaris
gtgtgcatggtcgggtcggggctgagcgaactaagccttggggaactttgcgc

SEQ ID NO: 130 - DNA, 53 bp, Canis lupus
  source 1..53: mol_type = genomic DNA; note = canis familiaris
ccctcgcctccccccacttctcatcccgaggaacagtcttgggatggaaagaa

SEQ ID NO: 131 - DNA, 53 bp, Canis lupus
  source 1..53: mol_type = genomic DNA; note = canis familiaris
actcggcaggtgctcaggtgggcactcggcgggcgctcaggcgggcactcggt

SEQ ID NO: 132 - DNA, 53 bp, Canis lupus
  source 1..53: mol_type = genomic DNA; note = canis familiaris
aaagaagcagaatcagcagcagccgccgcctccgcaacagccccagctgccag

SEQ ID NO: 133 - DNA, 53 bp, Canis lupus
  source 1..53: mol_type = genomic DNA; note = canis familiaris
ggttccttcacccttacacgggggcccgagtggccgtggcccccctgccagag

SEQ ID NO: 134 - DNA, 53 bp, Canis lupus
  source 1..53: mol_type = genomic DNA; note = canis familiaris
ttggcctcctcacctggagcttgggccgctctgataccctcctaggcaaaatt

SEQ ID NO: 135 - DNA, 53 bp, Canis lupus
  source 1..53: mol_type = genomic DNA; note = canis familiaris
cacagcccgcagagcctttcacttctcgggggatgatgtcgctcactcctaga

SEQ ID NO: 136 - DNA, 53 bp, Canis lupus
  source 1..53: mol_type = genomic DNA; note = canis familiaris
cgaattgccccggcccgtctcaagttcgcacgcaggctgctgggttccgtgtc

SEQ ID NO: 137 - DNA, 53 bp, Canis lupus
  source 1..53: mol_type = genomic DNA; note = canis familiaris
acccttgggtttttgtcaacagcaaccggaggtgacgttcttttctcacactt

SEQ ID NO: 138 - DNA, 53 bp, Canis lupus
  source 1..53: mol_type = genomic DNA; note = canis familiaris
tgcagcagcgccgtcttgccgcactccgcgtcccccaccaccacgatcttgca

SEQ ID NO: 139 - DNA, 53 bp, Canis lupus
  source 1..53: mol_type = genomic DNA; note = canis familiaris
gtgagttcactgtatgtcagccagcacgtcagagccagcgtctcttctcaccc

SEQ ID NO: 140 - DNA, 53 bp, Canis lupus
  source 1..53: mol_type = genomic DNA; note = canis familiaris
ctccaaaaccccaggcaggtgggatccgacagtcaagatctgtccccttccaa

SEQ ID NO: 141 - DNA, 53 bp, Canis lupus
  source 1..53: mol_type = genomic DNA; note = canis familiaris
gccacccaagggtgtgccgcagccagcgcaggaagaacgagaagtttcctgca

SEQ ID NO: 142 - DNA, 53 bp, Canis lupus
  source 1..53: mol_type = genomic DNA; note = canis familiaris
cctacctaactcgatcccgtcctcaccgcaggctcctacctaactcgatcctg

SEQ ID NO: 143 - DNA, 53 bp, Canis lupus
  source 1..53: mol_type = genomic DNA; note = canis familiaris
ttgcgaccgatgacctgtgaattggccgaaggagctgtgactcctggatttgg

SEQ ID NO: 144 - DNA, 53 bp, Canis lupus
  source 1..53: mol_type = genomic DNA; note = canis familiaris
ctctttctccttctgcacaagggagacggccaataaacaacagtgagtttgcc

SEQ ID NO: 145 - DNA, 53 bp, Canis lupus
  source 1..53: mol_type = genomic DNA; note = canis familiaris
accctgcagcacagcgtccccttccccgcggccgaggaacgagcgccggaacc

SEQ ID NO: 146 - DNA, 53 bp, Canis lupus
  source 1..53: mol_type = genomic DNA; note = canis familiaris
aattccaccccaaccccaggagctcccggccccacggaagagacagtacagcg

SEQ ID NO: 147 - DNA, 53 bp, Canis lupus
  source 1..53: mol_type = genomic DNA; note = canis familiaris
cctacctcctcggacagcatcgcccccgaccccgcgagccctgagcgctcccc

SEQ ID NO: 148 - DNA, 53 bp, Canis lupus
  source 1..53: mol_type = genomic DNA; note = canis familiaris
actcggcgggggctcaggtgggcactcggcaggtgctcaggtgggcactcggc

SEQ ID NO: 149 - DNA, 53 bp, Canis lupus
  source 1..53: mol_type = genomic DNA; note = canis familiaris
ggaggcctcggaaacagcggagaacacggtgacctttcaaggagatgcaggga

SEQ ID NO: 150 - DNA, 53 bp, Canis lupus
  source 1..53: mol_type = genomic DNA; note = canis familiaris
gcggcctttggagtgactcccatctccgtgccagccgccggacttgggggccg

SEQ ID NO: 151 - DNA, 53 bp, Canis lupus
  source 1..53: mol_type = genomic DNA; note = canis familiaris
ccacaagatcaacaaagaatctatcccgttcttccagcccatcgccaaggcct

SEQ ID NO: 152 - DNA, 53 bp, Canis lupus
  source 1..53: mol_type = genomic DNA; note = canis familiaris
taccacctgtcatcacccacggggctcggtgcccaggccgggggcatcactga

SEQ ID NO: 153 - DNA, 53 bp, Canis lupus
  source 1..53: mol_type = genomic DNA; note = canis familiaris
tactagatgcaacctagggtcgcaaccggttgttagaaacaacactaatacat

SEQ ID NO: 154 - DNA, 53 bp, Canis lupus
  source 1..53: mol_type = genomic DNA; note = canis familiaris
acacgcccgggtagggtacatgacgccgggggccaggggcgcggaaagcagag

SEQ ID NO: 155 - DNA, 53 bp, Canis lupus
  source 1..53: mol_type = genomic DNA; note = canis familiaris
gcagcggggcccagcagcgccgagggcggccaccccatcagtggggccaccgc

SEQ ID NO: 156 - DNA, 53 bp, Canis lupus
  source 1..53: mol_type = genomic DNA; note = canis familiaris
ctctgcgctggggccccgggcggctccgctgctcccccgccccaggggagtct

SEQ ID NO: 157 - DNA, 53 bp, Canis lupus
  source 1..53: mol_type = genomic DNA; note = canis familiaris
actgcggttggctgagcagatgcctacggtattttggacacaaattacagaaa

SEQ ID NO: 158 - DNA, 53 bp, Canis lupus
  source 1..53: mol_type = genomic DNA; note = canis familiaris
cccgagggctggaagagtgtcctccccgcgagcggggctcctggcggggggcg

SEQ ID NO: 159 - DNA, 53 bp, Canis lupus
  source 1..53: mol_type = genomic DNA; note = canis familiaris
tgtgcgtgcaggctgcggcgagtgtgcgtgctcggctgggacctgagtgtaca

SEQ ID NO: 160 - DNA, 53 bp, Canis lupus
  source 1..53: mol_type = genomic DNA; note = canis familiaris
aagggccaaggccgctcagaacaagtcgcattccctgagggcttgatcatgcg

SEQ ID NO: 161 - DNA, 53 bp, Canis lupus
  source 1..53: mol_type = genomic DNA; note = canis familiaris
ggggaggcgccgtcggggcccccagccggctgccggtgcagcctgccccacgg

SEQ ID NO: 162 - DNA, 53 bp, Canis lupus
  source 1..53: mol_type = genomic DNA; note = canis familiaris
tcctagtggtgcaccctctggctcaccgcctccacgttagacctcacccccat

SEQ ID NO: 163 - DNA, 53 bp, Canis lupus
  source 1..53: mol_type = genomic DNA; note = canis familiaris
atcatctacctcatctgcagtcacagcgagctccacaagcccatgtacttctt

SEQ ID NO: 164 - DNA, 53 bp, Canis lupus
  source 1..53: mol_type = genomic DNA; note = canis familiaris
ttggacgtcagggggcggatctgcgccgggcctttcgcgtccaggggctggga

SEQ ID NO: 165 - DNA, 53 bp, Canis lupus
  source 1..53: mol_type = genomic DNA; note = canis familiaris
gattttcatgccacgttctctaactacgtttgaagttaggcaggcagggctta

SEQ ID NO: 166 - DNA, 53 bp, Canis lupus
  source 1..53: mol_type = genomic DNA; note = canis familiaris
ggtcagggcctgtgagtgtgcatggtcgggtcggggctgagcgaactaagcct

SEQ ID NO: 167 - DNA, 53 bp, Canis lupus
  source 1..53: mol_type = genomic DNA; note = canis familiaris
tcacggtctcagctatgacgagggcgcgaaacccctcgatgtcctcccactct

SEQ ID NO: 168 - DNA, 53 bp, Canis lupus
  source 1..53: mol_type = genomic DNA; note = canis familiaris
gtggggggtgtatgtggtgggaacggcgcggtgctggggcccggaggcctccc

SEQ ID NO: 169 - DNA, 53 bp, Canis lupus
  source 1..53: mol_type = genomic DNA; note = canis familiaris
aactccagtcacatgggtctgccttccgtcccctgcacactgtccggtctccc

SEQ ID NO: 170 - DNA, 53 bp, Canis lupus
  source 1..53: mol_type = genomic DNA; note = canis familiaris
acaccgcctgcaagtcctggcaccgccgcccccttccattttcacaaaggctc

SEQ ID NO: 171 - DNA, 53 bp, Canis lupus
  source 1..53: mol_type = genomic DNA; note = canis familiaris
gtcctcactgcaggctccaactgactcgatcccgtcctcaccgcaggctccta

SEQ ID NO: 172 - DNA, 53 bp, Canis lupus
  source 1..53: mol_type = genomic DNA; note = canis familiaris
aagctaatcccacacaccagtgtgcccgcagtagctgcagctgggcctctcag

SEQ ID NO: 173 - DNA, 53 bp, Canis lupus
  source 1..53: mol_type = genomic DNA; note = canis familiaris
ctctgcaccccagctcctccgcaccccggctcgcggtcaggagatgcccagga

SEQ ID NO: 174 - DNA, 53 bp, Canis lupus
  source 1..53: mol_type = genomic DNA; note = canis familiaris
agccccttccaaaaccacactgtgcccggtccagctcctctgccatgtgcccc

SEQ ID NO: 175 - DNA, 53 bp, Canis lupus
  source 1..53: mol_type = genomic DNA; note = canis familiaris
gtctccaggccccccaggggctccagcgctcgcaggaggacgcacgccccatc

SEQ ID NO: 176 - DNA, 53 bp, Canis lupus
  source 1..53: mol_type = genomic DNA; note = canis familiaris
ggcccgcggccttcgggcgggcaagccgcgggactgacaggccagcacagtgg

SEQ ID NO: 177 - DNA, 53 bp, Canis lupus
  source 1..53: mol_type = genomic DNA; note = canis familiaris
caggggactccttgcttggagtttaacggtcgagacagcacaggaatgagcac

SEQ ID NO: 178 - DNA, 53 bp, Canis lupus
  source 1..53: mol_type = genomic DNA; note = canis familiaris
gtgcggcgaccgtgtgaccgtgtccccgcgagcaggtgcgctcgtccagggcg

SEQ ID NO: 179 - DNA, 53 bp, Canis lupus
  source 1..53: mol_type = genomic DNA; note = canis familiaris
gggcccagaaatgcggcctcccctcccgcagccacagcccccgtagccagccc

SEQ ID NO: 180 - DNA, 53 bp, Canis lupus
  source 1..53: mol_type = genomic DNA; note = canis familiaris
cagccagtgaggctgctgatcaaggtcggccagggaggatgctgatcaaggtt

SEQ ID NO: 181 - DNA, 53 bp, Canis lupus
  source 1..53: mol_type = genomic DNA; note = canis familiaris
tcattttcttcctccccgtcccgtcccgtcccctctgggacagaccacagcag

SEQ ID NO: 182 - DNA, 53 bp, Canis lupus
  source 1..53: mol_type = genomic DNA; note = canis familiaris
gcctgtgtcccctcagccaccgtgatcgtaacgaattatagtggcggaggata

SEQ ID NO: 183 - DNA, 53 bp, Canis lupus
  source 1..53: mol_type = genomic DNA; note = canis familiaris
ctgggcaggagctcggatctgaagtccggtgttggcggggcgggcatcagcgg

SEQ ID NO: 184 - DNA, 53 bp, Canis lupus
  source 1..53: mol_type = genomic DNA; note = canis familiaris
tcaccctccccagggcatgccagggccggcctcaaaggggaacgtgaaaggaa

SEQ ID NO: 185 - DNA, 53 bp, Canis lupus
  source 1..53: mol_type = genomic DNA; note = canis familiaris
gtcctttcccggcttctggaggcgcccgcgttcctgggctcgggcgctccccg

SEQ ID NO: 186 - DNA, 53 bp, Canis lupus
  source 1..53: mol_type = genomic DNA; note = canis familiaris
gcggcctccgccccaaccagacgcggcgcgcccgggacgcagggggcggttct

SEQ ID NO: 187 - DNA, 53 bp, Canis lupus
  source 1..53: mol_type = genomic DNA; note = canis familiaris
tcagctcgcctcttagactcatacaacgcaaggaactgaggacggggacatct

SEQ ID NO: 188 - DNA, 53 bp, Canis lupus
  source 1..53: mol_type = genomic DNA; note = canis familiaris
ctcccccggagtcaaagatcttgacccgcctcctgccgtcgtgcaccaccacg

SEQ ID NO: 189 - DNA, 53 bp, Canis lupus
  source 1..53: mol_type = genomic DNA; note = canis familiaris
gccaccatcttgtgaggcgagcagggcgtcgaggccggcgggtgagggggcgc

SEQ ID NO: 190 - DNA, 53 bp, Canis lupus
  source 1..53: mol_type = genomic DNA; note = canis familiaris
tgtgcgtgcaggctgcggcgagtgtgcgtgcaggctggggctggggctgagtg

SEQ ID NO: 191 - DNA, 53 bp, Canis lupus
  source 1..53: mol_type = genomic DNA; note = canis familiaris
ataccaggcgactgcgctcaaggtctcggggtaaaggccagcgtctgggacgt

SEQ ID NO: 192 - DNA, 53 bp, Canis lupus
  source 1..53: mol_type = genomic DNA; note = canis familiaris
ccccaggtagctccccttcctgtcagcgtgcgggtgcgtggggggcatccagg

SEQ ID NO: 193 - DNA, 53 bp, Canis lupus
  source 1..53: mol_type = genomic DNA; note = canis familiaris
gtgtagggagacccttcatcctcagacgaccctcaaaacacatacctccccaa

SEQ ID NO: 194 - DNA, 53 bp, Canis lupus
  source 1..53: mol_type = genomic DNA; note = canis familiaris
gggcacttggcaggcactaaggcaggcgctcaggtgggtacttagtgggtgct

SEQ ID NO: 195 - DNA, 53 bp, Canis lupus
  source 1..53: mol_type = genomic DNA; note = canis familiaris
tcactcgggctatgaaacacatcgtgcgcaaaacaccacccgccgggcctgta

SEQ ID NO: 196 - DNA, 53 bp, Canis lupus
  source 1..53: mol_type = genomic DNA; note = canis familiaris
acagcgcgcttcgccaggccgagccccgctgctttccaagaccacccccctca

SEQ ID NO: 197 - DNA, 53 bp, Canis lupus
  source 1..53: mol_type = genomic DNA; note = canis familiaris
tgtgcatctggggcccggtgtgagtgcgagacctgataggtgtgtggggccca

SEQ ID NO: 198 - DNA, 53 bp, Canis lupus
  source 1..53: mol_type = genomic DNA; note = canis familiaris
atgccagggccggcctcaaaggggaacgtgaaaggaacaccagttatcccatg

SEQ ID NO: 199 - DNA, 53 bp, Canis lupus
  source 1..53: mol_type = genomic DNA; note = canis familiaris
cccgcggccttgcctcggccacgctccgcccgaggggtccccgtgggctgggt

SEQ ID NO: 200 - DNA, 53 bp, Canis lupus
  source 1..53: mol_type = genomic DNA; note = canis familiaris
tgctgggagccccagggggtgtctgacggtcaccctgggttgtcaccacccta